(19) United States
(12) Patent Application Publication
(10) Pub. No.: US 2006/0141537 A1
(43) Pub. Date: Jun. 29, 2006

(54) Device and method for separating molecules

(75) Inventors: Laurie Engel, DeForest, WI (US); Tonny Johnson, Madison, WI (US)

Correspondence Address: MICHAEL BEST & FRIEDRICH, LLP, ONE SOUTH PINCKNEY STREET, P O BOX 1806, MADISON, WI, 53701, US

(73) Assignee: Promega Corporation, Madison, WI (US)

(21) Appl. No.: 11/358,519

(22) Filed: Feb. 21, 2006

Related U.S. Application Data
(62) Division of application No. 11/029,882, filed on Jan. 5, 2005.
Application No. 11/029,882 is a continuation-in-part of application No. 10/987,514, filed on Nov. 12, 2004.

(51) Int. Cl. G01N 33/53

(52) U.S. Cl. 435/7.1

(57) ABSTRACT

An apparatus and method for separating a biomolecule from one or more contaminants in a sample. The apparatus can include a unitary device comprising a plurality of fractionation devices, each reservoir comprising a filter, each reservoir adapted to contain a solid phase, the solid phase adapted to separate the biomolecule and the contaminant by fractionation. The filter can have an average pore size that allows the sample to pass therethrough while substantially preventing the solid phase from passing therethrough. The method can include moving the sample past the solid phase in each reservoir to separate the biomolecule from the contaminant by fractionation to obtain an isolated biomolecule.

### CROSS-REFERENCE TO RELATED APPLICATIONS

This application is a divisional of U.S. patent application Ser. No. 11/029,882 filed Jan. 5, 2005, which is a continuation-in-part of U.S. patent application Ser. No. 10/987,514 filed Nov. 12, 2004, and is incorporated herein by reference in its entirety.

### STATEMENT OF FEDERALLY SPONSORED RESEARCH AND DEVELOPMENT

Not applicable.

### BACKGROUND OF THE INVENTION

Generally, in systems for isolating a biomolecule from a complex biological material, insoluble matter is initially removed from a sample using a known technique, such as some type of filtration, centrifugation or other separation method. After the insoluble matter has been removed from the sample, the sample includes the biomolecule of interest and other soluble matter. Some type of solid phase or other material used to capture the biomolecule of interest can then be added to the soluble matter of the sample to form a biomolecule-solid phase complex. Again, a known separation method such as filtration or centrifugation can be used to isolate the biomolecule-solid phase complex from the other soluble matter of the sample. Finally, the biomolecule of interest can be removed from the solid phase to isolate the biomolecule of interest. Generally, these systems require initial removal of any insoluble matter from the sample before the sample can be combined with any solid phase.

### SUMMARY OF THE INVENTION

Some embodiments of the present invention provide a method of isolating a biomolecule. The method comprises: providing a sample comprising the biomolecule and insoluble matter; providing a reservoir comprising a filter, the reservoir adapted to contain a solid phase, the solid phase adapted to capture the biomolecule; adding the sample to the reservoir; combining the sample with the solid phase; and removing the insoluble matter from the sample by passing the insoluble matter through the filter, the filter having an average pore size sufficiently small to substantially prevent the solid phase from passing therethrough.

In some embodiments of the present invention, an apparatus for isolating a biomolecule from a sample is provided. The sample comprises the biomolecule and insoluble matter. The apparatus comprises: a reservoir comprising a filter, the reservoir adapted to contain a solid phase, the solid phase adapted to capture the biomolecule; the filter having an average pore size that allows the insoluble matter to pass therethrough while substantially preventing the solid phase from passing therethrough.

Some embodiments of the present invention provide a kit for isolating a biomolecule from a sample, the sample comprising the biomolecule and insoluble matter. The kit comprises: a plurality of first reservoirs, each first reservoir comprising a filter; a solid phase adapted to capture the biomolecule, the solid phase contained at least partially within each first reservoir; the filter having an average pore size that allows the insoluble matter to pass therethrough while substantially preventing the solid phase from passing therethrough.

In some embodiments of the present invention, an apparatus for isolating a biomolecule from a sample is provided. The sample comprises the biomolecule and insoluble matter. The apparatus comprises: a solid phase adapted to capture the biomolecule; a reservoir comprising an inner surface, the reservoir adapted to contain the sample and the solid phase; and a filter positioned between the solid phase and at least a portion of the inner surface of the reservoir, the filter adapted to inhibit passage of the solid phase while allowing passage of the insoluble matter.

Some embodiments of the present invention provide a method of isolating a biomolecule from a sample, the sample comprising the biomolecule and insoluble matter. The method comprises: providing a reservoir comprising an inner surface, the reservoir adapted to contain the sample, the inner surface comprising a solid phase adapted to capture the biomolecule; adding the sample to the reservoir to allow the solid phase to capture the biomolecule; removing the insoluble matter from the sample; and removing the biomolecule from the solid phase.

In some embodiments of the present invention, an apparatus for isolating a biomolecule from a sample is provided. The sample comprises the biomolecule and insoluble matter. The apparatus comprises: a reservoir comprising an inner surface, the inner surface comprising a solid phase adapted to capture the biomolecule; and an aperture defined in the inner surface of the reservoir, the aperture adapted to allow removal of the insoluble matter from the reservoir.

Some embodiments of the present invention provide a method of isolating a biomolecule. The method comprises: providing a sample comprising the biomolecule and insoluble matter; combining the sample with a solid phase, the solid phase being adapted to capture the biomolecule; removing the insoluble matter from the sample; and removing the biomolecule from the solid phase.

Some embodiments of the present invention provide a method for isolating a biomolecule from a sample, the method comprising: providing a reservoir comprising a filter, the reservoir adapted to contain a solid phase, the solid phase adapted to capture the biomolecule; combining the solid phase with the sample; extracting the biomolecule from the sample substantially simultaneously with combining the solid phase with the sample; capturing the biomolecule with the solid phase; and removing uncaptured matter from the sample by passing the uncaptured matter through the filter, the filter having an average pore size sufficiently small to substantially prevent the solid phase from passing therethrough.

In some embodiments of the present invention, an apparatus for isolating a biomolecule from a sample is provided. The sample comprises the biomolecule and insoluble matter. The apparatus comprises: a reservoir comprising an inner surface, the reservoir adapted to at least partially contain the sample; means for capturing the biomolecule; and at least one of: a filter positioned between the means for capturing the biomolecule and at least a portion of the inner surface of the reservoir, the filter adapted to inhibit passage of the means for capturing the biomolecule therethrough while allowing for passage of the insoluble matter therethrough, and an aperture defined in the inner surface of the reservoir, the aperture adapted to allow the insoluble matter to be removed from the reservoir.

Some embodiments of the present invention provide a method for isolating a biomolecule from a sample, the sample comprising the biomolecule and a contaminant. The method can include providing a unitary device including a plurality of fractionation devices, each fractionation device including a reservoir. The reservoir can include a filter and can be adapted to contain a solid phase. The solid phase can be adapted to separate the biomolecule from the contaminant. The filter can be adapted to inhibit passage of the solid phase therethrough while allowing passage of the sample therethrough. The method can further include moving the sample past the solid phase in the plurality of fractionation devices to separate the biomolecule from the contaminant by fractionation to obtain an isolated biomolecule.

In some embodiments of the present invention, an apparatus for isolating a biomolecule from a sample is provided. The sample can include a biomolecule and a contaminant. The apparatus can include a unitary device including a plurality of fractionation devices, each fractionation device including a reservoir, the reservoir including a filter. The reservoir can be adapted to contain a solid phase, and the solid phase can be adapted to separate the biomolecule and the contaminant by fractionation. The filter can have an average pore size that allows the sample to pass therethrough while substantially preventing the solid phase from passing therethrough.

Some embodiments of the present invention provide a method of isolating a biomolecule. The method can include providing a sample comprising the biomolecule and insoluble matter; providing a first reservoir comprising a filter, the first reservoir adapted to contain a first solid phase, the first solid phase adapted to capture the biomolecule; adding the sample to the first reservoir; combining the sample with the first solid phase; removing the insoluble matter from the sample by passing the insoluble matter through the filter, the filter having an average pore size sufficiently small to substantially prevent the first solid phase from passing therethrough; contacting the biomolecule and the first solid phase with an elution buffer to form an eluate comprising the biomolecule and a contaminant; passing the eluate through the filter; adding the eluate to a second reservoir, the second reservoir adapted to contain a second solid phase, the second solid phase adapted to separate the biomolecule and the contaminant; moving the eluate past the second solid phase; and separating the biomolecule and the contaminant by fractionation to obtain an isolated biomolecule.

In some embodiments of the present invention, an apparatus for isolating a biomolecule from a sample is provided. The apparatus can include a first reservoir comprising a first filter, the reservoir adapted to contain a first solid phase, the first solid phase adapted to capture the biomolecule, the first filter having an average pore size that allows the insoluble matter to pass therethrough while substantially preventing the solid phase from passing therethrough; and a second reservoir comprising a second filter, the second reservoir adapted to contain a second solid phase and an eluate eluted from the first solid phase and passed through the first filter, the eluate comprising the biomolecule and a contaminant, the second solid phase adapted to separate the biomolecule and the contaminant, the second filter having an average pore size that allows at least one of the biomolecule and the contaminant to pass therethrough while preventing the second solid phase from passing therethrough.

Other features and aspects of the invention will become apparent to those skilled in the art upon review of the following detailed description, claims and drawings.

### BRIEF DESCRIPTION OF THE DRAWINGS

FIG. 1 is a partial, perspective view of one embodiment of a biomolecule isolation apparatus according to the present invention, showing a biomolecule interacting with a solid phase.

FIG. 2 is a partial cross-sectional view of the apparatus of FIG. 1 taken along line 2-2.

FIG. 3 is a schematic view of the apparatus of FIGS. 1 and 2, showing removal of the biomolecule from the solid phase.

FIG. 4 is a schematic view of another embodiment of a biomolecule isolation apparatus according to the present invention, showing a biomolecule being captured from a sample by a solid phase.

FIGS. 5A-5C illustrate a biomolecule isolation system and method according to one embodiment of the present invention.

FIG. 6 is a side view of another embodiment of a biomolecule isolation apparatus according to the present invention.

FIG. 7 is a cross-sectional view of another embodiment of a biomolecule isolation apparatus according to the present invention.

FIG. 8 is a cross-sectional view of another embodiment of a biomolecule isolation apparatus according to the present invention.

FIG. 9 is a cross-sectional view of another embodiment of a biomolecule isolation apparatus according to the present invention.

FIG. 10 is a schematic view of another embodiment of a biomolecule isolation apparatus according to the present invention.

FIG. 11 is an electrophoretic gel showing automated purification of 6× Histidine-tagged firefly luciferase from BL-21 (DE3) using a 25 μm frit as the filter.

FIG. 12 is an electrophoretic gel showing automated purification of 6× Histidine-tagged MAP-kinase (MAPK) from BL-21 (DE3) using a 90 μm mesh as the filter.

FIG. 13 is an electrophoretic gel showing automated purification of 6× Histidine-tagged Calmodulin from BL-21 (DE3) using a 90 μm mesh as the filter.

FIG. 14 is an electrophoretic gel showing manual purification of 6× Histidine-tagged firefly luciferase from BL-21 (DE3) using a 90 μm mesh as the filter.

FIG. 15 is an electrophoretic gel showing manual purification of 6× Histidine-tagged firefly luciferase from BL-21 (DE3) using a 25 μm frit as the filter.

FIG. 16 is a contaminant removal system and method according to one embodiment of the present invention.

FIG. 17 is a cross-sectional view of a fractionation device according to one embodiment of the present invention.

FIG. 18 is a contaminant removal system and method according to another embodiment of the present invention.

FIG. 19 is an image of a portion of a 96-well plate that includes standard titrations of imidazole HCl from two different sources, in triplicate, and samples of polyhistidine tagged Luciferase after removal of imidazole using a fractionation device of the present invention, all stained with COOMASSIE PLUS™ Protein Assay Reagent.

FIG. 20 is a graph showing a relationship between Luciferase activity (as measured by luminescence) and volume of fractionation solid phase used to separate Luciferase and imidazole.

FIG. 21 is an electrophoretic gel showing the polyhistidine tagged Luciferase after being separated from imidazole using various volumes of fractionation solid phase.

Before any embodiments of the present invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings.

### DETAILED DESCRIPTION

The present invention is generally directed to a device, method and kit for isolating a biomolecule from a sample.

As used herein and in the appended claims, the term “complex biological material” refers to a biological material, or derivatives thereof, that occurs in or is formed by a living organism (i.e., a prokaryote, a eukaryote, a virus, or an organism from any other kingdom of life), and includes insoluble matter. For example, a “complex biological material” can include, without limitation, at least one of cell lysate, blood, urine, feces, cells, tissues, organs, plant materials, food sources, water, soil, and combinations thereof.

As used herein and in the appended claims, the term “solid phase” refers to a material that is selected to capture a biomolecule of interest from a sample (e.g., a complex biological material) as a result of combining the sample and the solid phase.

As used herein and in the appended claims, the term “biomolecule” refers to a molecule, or a derivative thereof, that occurs in or is formed by a living organism (i.e., a prokaryote, a eukaryote, a virus, or an organism from any other kingdom of life). For example, a biomolecule can include, without limitation, at least one of an amino acid, a nucleic acid, a polypeptide, a polynucleotide, a lipid, a phospholipid, a saccharide, a polysaccharide, and combinations thereof. Furthermore, a biomolecule can include, without limitation, at least one of mRNA, total RNA, genomic DNA, plasmid DNA, plant DNA, a GST fusion protein, a Histidine (His) tagged protein, an antibody, an antigen, and combinations thereof.

As used herein and in the appended claims, the terms “soluble matter” and “insoluble matter” refer to matter that is relatively soluble or insoluble in a given medium, under certain conditions. Specifically, under a given set of conditions, “soluble matter” is matter that goes into solution and can be dissolved in the solvent of the system. “Insoluble matter” is matter that, under a given set of conditions, does not go into solution and is not dissolved in the solvent of the system.

As used herein and in the appended claims, the term “fractionation” refers to a process by which a biomolecule of interest and one or more contaminants are separated into different portions or fractions such that the concentration of the biomolecule relative to that of the contaminant in one or more fractions is greater than that of the concentration of the biomolecule relative to that of the contaminant in the sample (e.g., the eluate transferred from the second reservoirs 120 of the biolmolecule isolation system 150) Specifically, fractionation can include at least one of size exclusion chromatography, gel filtration, molecular sieve chromatography, ion exchange chromatography, affinity chromatography, and combinations thereof.

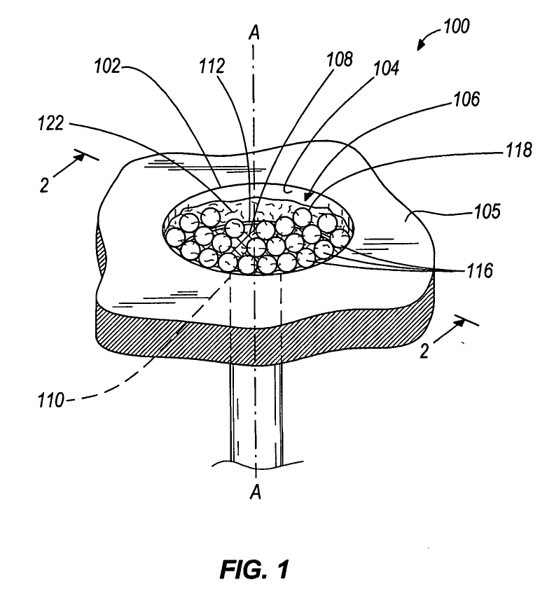

FIGS. 1-3 illustrate a biomolecule isolation apparatus 100 that includes a reservoir 102 having an inner surface 104, a solid phase 106 contained within the reservoir 102 and adapted to capture a biomolecule 122 from a sample, a filter 108 positioned between the solid phase 106 and at least a portion of the inner surface 104, a seal-forming device 112 (e.g., an o-ring) positioned adjacent the periphery of the filter 108 and a portion of the inner surface 104 to maintain an adequate seal around the periphery of the filter 108, and an aperture 110 defined in the inner surface 104 of the reservoir 102.

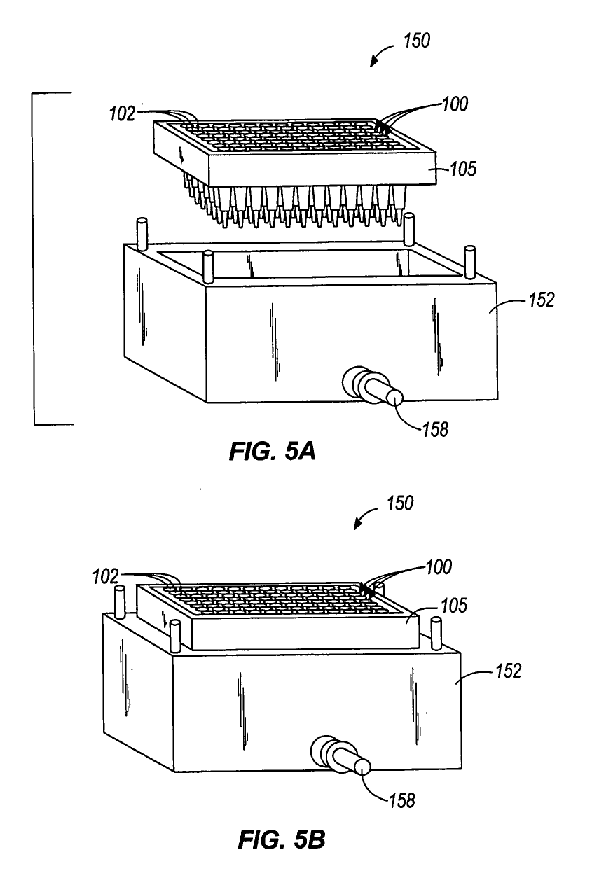

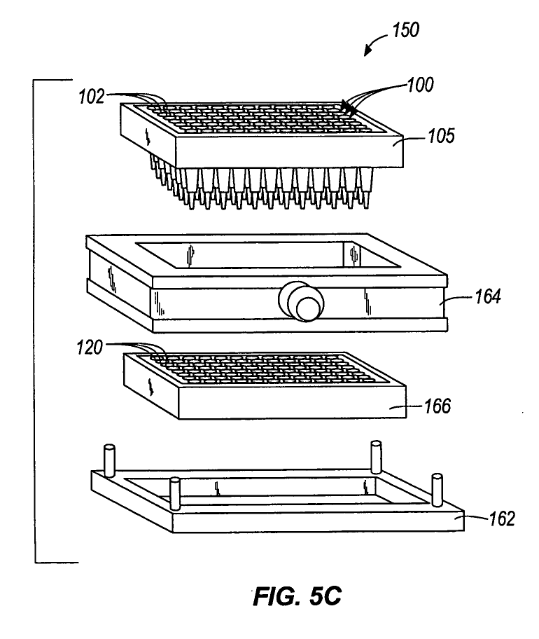

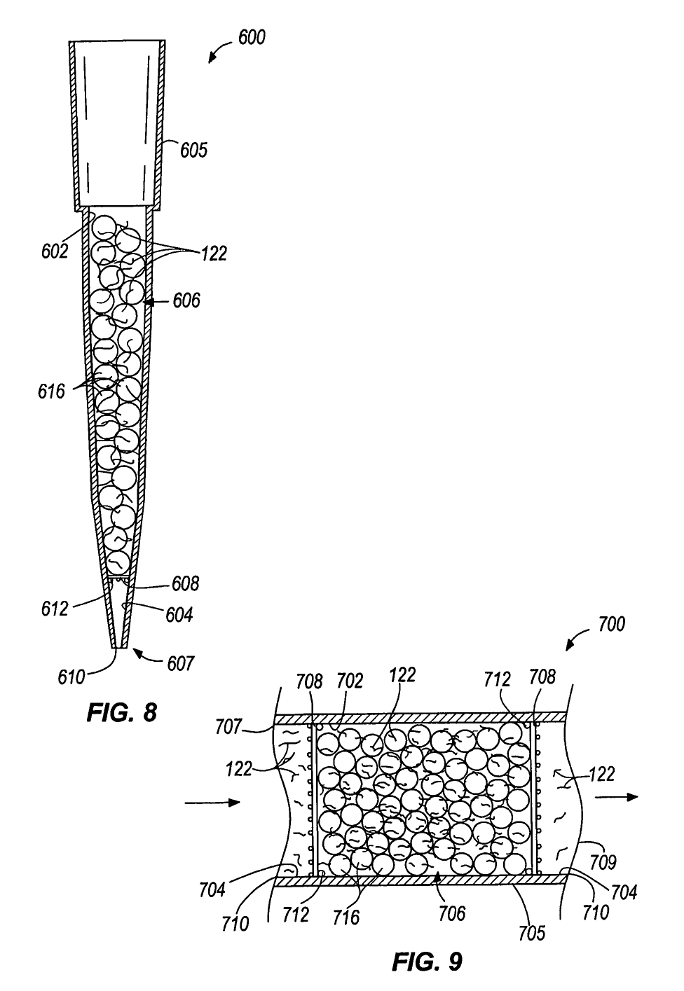

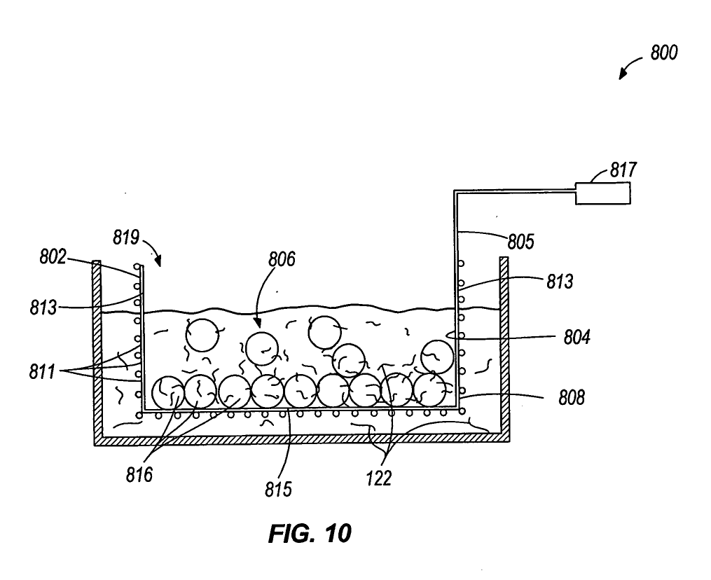

The reservoir 102 can be one of a plurality of reservoirs 102 in the biomolecule isolation apparatus 100, and can be at least partially defined by a multi-well plate 105 (as illustrated in FIGS. 5A-5C and described in greater detail below), a pipette tip 605 (as illustrated in FIG. 8 and described in greater detail below), a capillary column 705 (as illustrated in FIG. 9 and described in greater detail below), a basket 805 (as illustrated in FIG. 10 and described in greater detail below) and combinations thereof.

The reservoir 102 illustrated in FIGS. 1-3 is defined by a well of a multi-well plate 105 (e.g., a 96-well tissue culture plate, as is well-known in the art). The reservoir 102 illustrated in FIGS. 1-3 has a generally cylindrical shape with a generally uniform cross-section. However, it should be understood that cross-section of the reservoir 102 is not necessarily circular or uniform, and can taper toward an upper end 114 and/or a lower end 115. The reservoir 102 can have a variety of other shapes, including without limitation, hemispherical, conical, frustoconical, box-shaped, etc., and combinations thereof.

The solid phase 106 illustrated in FIGS. 1-3 includes a plurality of particles 116. However, it should be noted that as few as one particle 116 can be used with the present invention, and as many as structurally possible to be contained within the reservoir 102. Particularly, the amount of particles 116 used can depend on the desired amount of the biomolecule 122 of interest that is to be isolated. Each particle 116 is illustrated in FIGS. 1-3 as being generally spherical. However, any shape of particle 116 can be used without departing from the spirit and scope of the present invention. In addition, the porosity (e.g., as characterized by average pore volume, average pore size, total pore volume, etc.) and surface area of each particle 116 can be controlled to suit the biomolecule 122 of interest. For example, particles 116 that include nickel ions for isolating his tagged proteins can have an average pore size of approximately 1000 Å. One of skill in the art will recognize that many different particles 116 with varying parameters can be used with the present invention to isolate a variety of biomolecules from a variety of samples without departing from the spirit and scope of the present invention.

A variety of solid phases 106 can be used with the present invention to isolate a variety of biomolecules from a sample. As described in greater detail below, the solid phase 106 can be selected based on its ability to inherently capture a desired biomolecule, or the solid phase 106 can be modified to capture a desired biomolecule. As a result, a solid phase 106 that is adapted to capture a particular biomolecule 122 of interest can be inherently adapted to capture the biomolecule 122, or it can be modified to capture the biomolecule 122. The capacity of the solid phase 106 for capturing the biomolecule 122 of interest is generally greater than the amount of the biomolecule 122 that is to be isolated.

The solid phase 106 can be made of a variety of materials, as will be described in greater detail below, and can either be buoyant in a variety of solutions, or can settle in the reservoir 102. In some embodiments, the solid phase 106 is buoyant such that the sample can move freely about all outer surfaces of the solid phase 106. In some embodiments, the solid phase 106 can gravitationally settle in the reservoir 102, such that the sample can flow past the solid phase 106 that has settled in the reservoir 102. In some embodiments, the solid phase 106 can be formed of a combination of buoyant particles 116 and particles 116 that settle in the reservoir 102.

The filter 108 is positioned between at least a portion of the inner surface 104 of the reservoir 102 and the solid phase 106. The filter 108 allows matter from the sample that has not been captured by the solid phase 106 to be removed from the reservoir 102, while maintaining the solid phase 106 and the biomolecule 122 that has been captured from the sample by the solid phase 106 within the reservoir 102. As a result, the average pore size or mesh size of the filter 108 is at least partially determined by the size of the particles 116 in the solid phase 106. In addition, the average pore size or mesh size of the filter 108 is at least partially determined by the viscosity of the sample, and the size of any debris present in the sample. That is, the smaller the size of the particles 116, the smaller the average pore size or mesh size required by the filter 108 to retain the particles 116 of the solid phase 106 in the reservoir 102. However, the more viscous the sample, the larger the average pore size or mesh size required to allow passage of the matter in the sample that has not been captured by the solid phase 106. As a result, the average pore size or mesh size of the filter 108 needs to be adjusted to (1) maintain the solid phase 106 in the reservoir 102, and (2) allow the uncaptured matter in the sample to pass therethrough. The uncaptured matter can include any portion of the sample that was not captured by the solid phase 106, including insoluble matter, uncaptured biomolecules 122 of interest, other biomolecules present in the sample, etc. The filter 108 can include at least one of a woven mesh (e.g., a wire mesh, a cloth mesh, a plastic mesh, etc.), a sieve, an ablated film (e.g., a laser ablated film, a thermally ablated film, etc.), a punctured film, glass wool, a frit, filter paper, etc., and combinations thereof.

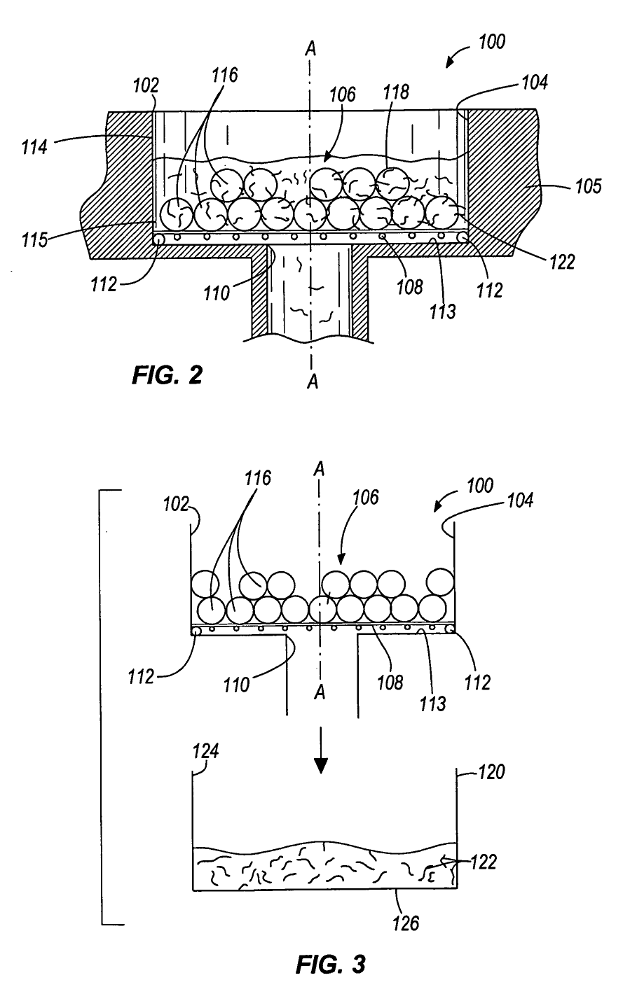

In some embodiments of the present invention, as illustrated in FIGS. 2-3, the filter 108 is positioned just above the seal-forming device 112 and disposed a small distance from a bottom surface 113 of the reservoir 102. However, in some embodiments, the filter 108 and seal-forming device 112 are positioned a greater distance from the bottom surface 113 of the reservoir 102. It should be noted that the seal-forming device 112 does not need to be positioned between the filter 108 and the bottom surface 113 of the reservoir 102. That is, in some embodiments, the seal-forming device 112 is positioned above the filter 108 in the reservoir 102. In some embodiments, the seal-forming device 112 is sandwiched between the periphery of the filter 108 and the inner surface 104 of the reservoir 102.

In some embodiments, as illustrated in the FIGS. 2-3, the reservoir 102 includes the bottom surface 113, and the cross-sectional size of the open upper end 114 of the reservoir 102 is greater than the cross-sectional size of the aperture 110 defined in the open lower end 115 of the reservoir 102. However, in some embodiments, the cross-sectional size of the open upper end 114 can be the same size as or smaller than the cross-sectional size of the open lower end 115. In such embodiments, the reservoir 102 does not include the bottom surface 113, and the filter 108 and seal-forming device 112 can be positioned at any vertical position in the reservoir 102.

In the embodiment illustrated in FIG. 1-3, the filter 108 is flat and positioned substantially perpendicularly with respect to a longitudinal axis A-A of the reservoir 102. In some embodiments, whether or not the reservoir 102 includes the bottom surface 113, the filter 108 is not flat, but instead is curved to fit adjacent any portion of the inner surface 104 of the reservoir, is wavy, or is positioned within the reservoir 102 at an angle other than 90° with respect to the longitudinal axis A-A. Any shape and orientation of filter 108 can be used without departing from the spirit and scope of the present invention.

In some embodiments, the biomolecule isolation apparatus 100 does not include the filter 108. For example, in some embodiments, the solid phase 106 includes one or more relatively large particles 116, and the particles 116 are sized such that the particles 116 will be retained in the reservoir 102 without the use of the filter 108. In such embodiments, one or more apertures 110 can be defined in the inner surface 104 of the reservoir 102 to allow insoluble matter to pass out of the reservoir 102 while retaining the solid phase 106 within the reservoir 102.

In some embodiments, the biomolecule isolation apparatus 100 does not include the aperture 110. That is, in some embodiments, the bottom surface 113 of the reservoir 102 is closed. In such embodiments, the insoluble matter (and any uncaptured matter) from the sample that is not captured by the solid phase 106 can be contained in the bottom of the reservoir 102, and the solid phase 106 with the captured biomolecule 122 can be transferred to another device for removal of the biomolecule 122 from the solid phase 106. That is, it is not required that the insoluble matter be completely removed from the reservoir 102, as long as the insoluble matter is separated from the solid phase 106 and the biomolecule 122 of interest without clogging.

The seal-forming device 112 can be formed of a variety of polymers, elastomers, composites, etc. The seal-forming device 112 can be a separate element from the reservoir 102, or the seal-forming device 112 can be integrally formed with the reservoir 102.

As mentioned above, the size of the particles 116 will at least partially depend on the biomolecule 122 to be isolated using the biomolecule isolation apparatus 100 of the present invention. In some embodiments, the particle size (i.e., the diameter of generally spherical particles 116) is greater than approximately 80 μm, particularly, greater than 100 μm, and more particularly, greater than approximately 120 μm. In addition, the particle size is less than approximately 240 μm, particularly, less than 220 μm, and more particularly, less than 200 μm. Accordingly, in embodiments employing the filter 108, the average pore size of the filter 108 can be less than approximately 200 μm, particularly, less than approximately 150 μm, and more particularly, less than approximately 100 μm. In addition, the average pore size of the filter 108 can be greater than approximately 75 μm, particularly, greater than approximately 90 μm (170 mesh size), and more particularly, greater than approximately 100 μm to allow proper removal of uncaptured material from the reservoir 102. The actual size of the particles 116 used and the average pore size of the filter 108 used will vary depending on the application (e.g., the type of complex biological material used, the biomolecule 122 of interest, the viscosity of the sample, etc.). One of ordinary skill in the art can easily alter the size of the particles 116 and the average pore size of the filter 108 to suit the application based on the relationships described above.

As mentioned above, the average pore size of the filter 108 can be at least partially dependent upon the viscosity of the sample. The viscosity of the sample can be at least partially dependent on cell number (particularly in embodiments in which the sample includes cells or cell lysate). Viscosity and cell number are at least partially dependent on several factors, including, without limitation, the type of media the cells are grown or incubated in, additives used in the media in which the cells are grown or incubated, temperature of the media (i.e., temperature at which the cells are grown or incubated), length of time the cells are grown or incubated, etc. For example, media including Terrific broth (TB) can lead to a three-fold increase in concentration (i.e., cell number) than media including Luria broth (LB), thereby leading to an increase in viscosity.

Nucleic acids, proteins and other macromolecules can be broken down (i.e., fragmented and/or hydrolyzed) to reduce the viscosity of the sample and increase the flow rate of the sample past the solid phase 106 by a variety of methods. Breaking down nucleic acids, proteins and other macromolecules in the sample can be accomplished using at least one of enzymatic methods, chemical (i.e., non-enzymatic) methods, mechanical methods, and combinations thereof to reduce viscosity and increase the flow rate of the sample past the solid phase 106 and out of the reservoir 102. Enzymatic methods can include, without limitation, adding enzymes, such as nucleases (e.g., DNases and RNases) and proteases, to the sample. Chemical methods can include, without limitation, adding at least one of Ce (IV), Pr(III), dicerium complex, phenazine di-N-oxide, magnesium(II) complex with diethylenetriamine, and combinations thereof to the sample. Mechanical methods can include, without limitation, at least one of sonication, using a French press, and combinations thereof. Reducing the viscosity of the sample also reduces the likelihood that the sample will clog the filter 108.

Additionally, warmer media will generally lead to a lower viscosity and a higher flow rate, as long as the increased temperature does not significantly disturb the properties of the sample or the interaction between the biomolecule 122 of interest and the solid phase 106.

Furthermore, if the viscosity of the sample is too low (i.e., the flow rate is accordingly too high to allow for sufficient interaction between the solid phase 106 and the sample), additives can be added to the sample to decrease the flow rate. Such additives can include, without limitation, at least one of macaloid clay, which can bind DNA and create a network; polyethylene glycols (PEGs); polyvinylpyrrolidones; ficcols; etc. Moreover, a colder media will generally lead to a higher viscosity and a slower flow rate, as long as the reduced temperature does not significantly disturb the properties of the sample or the interaction between the biomolecule 122 of interest and the solid phase 106.

In some embodiments of the present invention, and for particular samples and biomolecules of interest, a certain viscosity and associated flow rate is needed to achieve proper interaction or association between the biomolecule 122 of interest and the solid phase 106. That is, in some embodiments, if the sample is allowed to flow past the solid phase 106 and out of the reservoir 102 too quickly, the biomolecule 122 will not have been given an adequate time to interact with the solid phase 106, and will not be adequately isolated from the remainder of the sample. To achieve a certain flow rate for a particular sample, the viscosity of the sample can be increased or decreased, or the average pore size of the filter 108 can be increased or decreased.

In addition, in some embodiments, the sample can be incubated with the particles 116 of the solid phase 106 in a different container than the reservoir 102. This can be useful, for example, in situations where the flow rate of the sample through the reservoir 102 is too high to allow for sufficient interaction between the sample and the particles 116 (or another solid phase described below). The particles 116 of the solid phase 106 can be mixed with the sample for a period of time before adding the mixture of the particles 116 and the sample to the reservoir 102. The amount of time the sample is incubated with the particles 116 can vary depending on the application. Premixing the particles 116 with the sample can provide a facile method for enhancing the interaction between the sample and the particles 116. During incubation of the sample with the particles 116, the sample and particles 116 can be stirred, vortexed, shaken, etc. to enhance the interaction.

Furthermore, in embodiments in which the sample includes a lysate, the lysing step can occur substantially simultaneously with combining the sample with the particles 116 of the solid phase 106. That is, the biomolecule 122 of interest can be extracted from the sample, and the sample can be combined with the particles 116 (or another solid phase described below) without filtering, separating or purifying the sample between the extracting step and the combining step. In some embodiments, the particles 116 (or other solid phase, such as those described below) are combined with the sample prior to extracting the biomolecule 122 of interest from the sample. In some embodiments, the particles 116 are combined with the sample after extracting the biomolecule 122 of interest from the sample. In some embodiments, the particles 116 are combined with the sample at the same time as the biomolecule 122 of interest is extracted from the sample.

Various methods can be used to extract the biomolecule 122 of interest from the sample, depending on the complex biological material of the sample. For example, extracting can include lysing cells in the sample, increasing the permeability of cells in the sample (i.e., increasing the permeability of cell membranes and/or cell walls), and/or any other method that allows the particles 116 to capture the biomolecule 122 of interest, or that enhances the ability of the particles 116 to capture the biomolecule 122 of interest. Lysing cells can be accomplished using at least one of enzymatic methods, chemical (i.e., non-enzymatic) methods, mechanical methods, and combinations thereof. Enzymatic lysing methods can include, without limitation, adding at least one of lysozyme, pronase, and combinations thereof to the sample. Chemical lysing methods can include, without limitation, adding at least one of a detergent, a peptide (e.g., polymixinB), and combinations thereof to the sample. Mechanical lysing methods can include, without limitation, at least one of sonication, using a French press, and combinations thereof.

In addition, the particles 116 can capture the biomolecule 122 of interest from the sample substantially simultaneously with extracting the biomolecule 122 of interest and combining the sample with the particles 116. It should be understood that the extracting, combining and capturing steps can be performed sequentially and in different containers, but that performing these steps “substantially simultaneously” refers to performing these steps without any filtering, separating or purifying steps in between. Additionally, the viscosity of the sample can be increased or decreased (e.g., a nuclease can be added to the sample) substantially simultaneously with one or more of the extracting, combining and capturing steps.

With reference to FIGS. 1-3, a biomolecule 122 of interest can be isolated from any sample of a complex biological material using the biomolecule isolation apparatus 100. A sample that includes the biomolecule 122 of interest and insoluble matter can be combined with the solid phase 106 by adding the sample to the reservoir 102 and allowing the sample to interact with the solid phase 106. The solid phase 106 will be modified to, or inherently will, capture the biomolecule 122 of interest from the sample. The sample can further include other soluble matter that is not the biomolecule 122 of interest. The insoluble matter and any other soluble matter (which can include other biomolecules that are not of interest) present in the sample can be removed from the reservoir 102 via a variety of methods, including, without limitation, at least one of decanting, vacuum filtration, gravity filtration, centrifugation, etc., and combinations thereof. The embodiment illustrated in FIGS. 1-3 includes an aperture 110, such that any matter of the sample that is not captured by the solid phase 106 can be removed via the aperture 110.

FIGS. 1 and 2 schematically illustrate the solid phase 106 contained within the reservoir 102 by the filter 108, and several molecules of the biomolecule 122 of interest captured by the particles 116 of the solid phase 106. Specifically, the biomolecule 122 is shown as being captured by an outer surface 118 of the particles 116. In other embodiments, the biomolecule 122 can be captured within or encapsulated by a portion of the solid phase 106, as long as the biomolecule 122 can easily be removed from the solid phase 106 by a method known to those having ordinary skill in the art (e.g., elution, suction, trituration, agitation, etc.).

The biomolecule 122 can interact with the solid phase 106 by a variety of strong and weak interactions, including, without limitation, non-covalent bonding, such as ionic bonding, static charge interactions, hydrogen bonding, van der Waals interactions, protein-protein interactions, antibody-antigen bonding, DNA-DNA hybrids, RNA-DNA hybrids, oligonucleotide hybrids, etc., and combinations thereof.

FIG. 3 schematically illustrates several molecules of the biomolecule 122 after it has been removed from the solid phase 106 and the reservoir 102. Specifically, FIG. 3 illustrates a second reservoir 120. Similar to the reservoir 102, the second reservoir 120 can be defined at least partially by at least one of a multi-well plate (such as the second multi-well plate 166 illustrated in FIG. 5C and described below), a pipette tip, a capillary column, and combinations thereof. The second reservoir 120 is positioned such that the second reservoir 120 is in fluid communication with the reservoir 102 to receive the biomolecule 122 after it has been removed from the sample, the reservoir 102 and the solid phase 106. Specifically, the second reservoir 120 includes an open end 124 that is in fluid communication with the aperture 110 defined in the inner surface 104 of the reservoir 102. The second reservoir 120 further includes a closed end 126 such that the second reservoir 120 is adapted to contain the isolated biomolecule 122.

As mentioned above, the biomolecule 122 can be removed from the solid phase 106 by a variety of methods known in the art, including elution. That is, an elution solution that will disturb the interaction or association between the biomolecule 122 and the solid phase 106 can be added to the reservoir 102 and removed by any of the removal techniques mentioned above (i.e., decanting, vacuum filtration, gravity filtration, centrifugation, etc., and combinations thereof). The elution solution can be incubated for a predetermined period of time with the solid phase 106 in the reservoir 102. The elution step, or other removal technique, can be repeated one or more times to be sure that all of the biomolecule 122 has been removed from the solid phase 106. In addition, a washing solution can be added to the reservoir 102 in one or more washing steps (i.e., prior to the elution solution being added) to wash the solid phase 106, enhance removal, and increase yield of the biomolecule 122 from the solid phase 106. Repeated elution steps can be used to increase the yield of the isolated biomolecule, as is well-known to those of ordinary skill in the art.

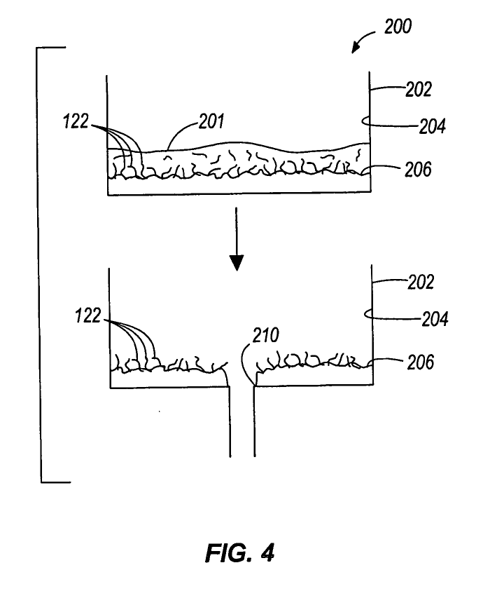

FIG. 4 illustrates a biomolecule isolation apparatus 200 according to another embodiment of the present invention. The biomolecule isolation apparatus 200 includes a reservoir 202 having an inner surface 204. The reservoir 202 illustrated in FIG. 4 is defined by a multi-well plate (not shown). At least a portion of the inner surface 204 includes a solid phase 206 adapted to capture a biomolecule 122 of interest from the sample. The portion of the inner surface 204 that includes the solid phase 206 can be textured, as illustrated in FIG. 4, such that the portion of the inner surface 204 that includes the solid phase 206 has an increased surface area to allow more biomolecules 122 of interest to interact with the solid phase 206. The textured inner surface 204 that acts as the solid phase 206 in the biomolecule isolation apparatus 200 can be formed of a material that inherently captures a biomolecule 122 of interest from a sample, or the textured inner surface 204 can be charged, coated or otherwise modified to capture the biomolecule 122 of interest.

In some embodiments, the portion of the inner surface 204 that includes the solid phase 206 can be defined by at least one of a woven mesh, a sieve, an ablated film, a punctured film, glass wool, a frit, filter paper, and combinations thereof. For example, a woven mesh can form at least a portion of the inner surface 204 of the reservoir 202, and accordingly, at least a portion of the solid phase 206. The woven mesh can be formed of a material that inherently captures a biomolecule 122 of interest from a sample, or the woven mesh can be charged, coated or otherwise modified to capture the biomolecule 122 of interest. For example, the solid phase 206 can be formed of a stainless steel mesh that is coated with positively-charged nickel ions to isolate his tagged proteins from a sample. In embodiments in which the solid phase 206 includes a woven mesh, the average pore size of the mesh would be set to control the flow rate of the sample through the mesh to allow proper time for the biomolecule 122 in the sample to interact with the solid phase 206.

In embodiments employing a textured inner surface 204 as the solid phase 206, as illustrated in FIG. 4, a sample 201 can be added to the reservoir 202 and contained within the reservoir 202. A biomolecule 122 of interest in the sample 201 is allowed to interact with the solid phase 206 integrally formed with the inner surface 204 of the reservoir 202 (whether the solid phase 206 is inherently part of the material forming the inner surface 204, or the inner surface 204 has been charged, coated or otherwise modified to include an immobilized solid phase 106 capable of capturing the biomolecule 122). After an adequate amount of time has passed to allow the biomolecule 122 to interact with the solid phase 206, the insoluble matter and any uncaptured, soluble matter in the sample can be removed from the reservoir 202. The insoluble matter, and any other uncaptured matter, can be removed from the sample 201 and the reservoir 202 using any of the removal techniques described above (i.e., decanting, vacuum filtration, gravity filtration, centrifugation, etc., and combinations thereof). In order to remove the uncaptured matter from the sample 201 and the reservoir 202, an aperture 210 can be defined in the inner surface 204 of the reservoir 202, as illustrated in FIG. 4. Depending on the size of aperture 201 needed, the aperture 210 can be defined in the inner surface 204 before or after the sample 201 is added to the reservoir 202. In the embodiment illustrated in FIG. 4, the aperture 210 is defined in the inner surface 204 after the sample 201 has been added to the reservoir 202. The aperture 210 can be defined in the inner surface 204 by a variety of techniques, including, without limitation, at least one of punching, puncturing, stamping, molding, drilling, etc., and combinations thereof.

In some embodiments of the present invention, the aperture 210 can be defined in the inner surface 204 throughout the biomolecule isolating process, and flow of the sample 201 through the aperture 210 can be controlled by any of a variety of valves (e.g., check valve, solenoid valve, etc.). In other embodiments, the aperture 210 can be mechanically and intermittently sealed. For example, a film covering can be positioned over the aperture 210 (e.g., a film covering can be positioned over at least a portion of a multi-well plate in which the reservoir 202 is defined), or a plug can be used to close the aperture 210 while the sample is allowed to interact with the solid phase 206 (e.g., a sheet with a plurality of plugs arranged to simultaneously plug one or more of the reservoirs 204 defined in a multi-well plate).

FIGS. 5A-5C illustrate one embodiment of a biomolecule isolation system 150 according to the present invention and a method for isolating a biomolecule from a sample using the biomolecule isolation system 150. The biomolecule isolation system 150 is shown by way of example only and is not intended to be limiting. The biomolecule isolation system 150 includes a vacuum manifold 152, and the multi-well plate 105, namely, the first multi-well plate 105 in the biomolecule isolation system 150. The first multi-well plate 105 includes a plurality of biomolecule isolation apparatuses 100, as described above and illustrated in FIGS. 1-3. Accordingly, the first multi-well plate 105 includes a plurality of reservoirs 102.

FIGS. 5A and 5B illustrate a separation setup for removal of the insoluble matter and any other uncaptured matter from a sample by vacuum filtration. FIG. 5A shows an exploded view of the separation setup, and FIG. 5B shows an assembled view. The first multi-well plate 105 fits adjacent the vacuum manifold 152 and is in fluid communication with an evacuation valve 158 in the vacuum manifold 152 to allow the reservoirs 102 of the first multi-well plate 105 to be evacuated.

The separation setup illustrated in FIGS. 5A and 5B can also be used for a washing step after the insoluble matter has been removed. During the washing step, a wash solution appropriate for a specific biomolecule-solid phase complex can be added to each of the reservoirs 102 and removed by vacuum filtration using the vacuum manifold 152. Particularly, the wash solution should not disrupt the interaction between the solid phase 106 and the sample, but should enhance the removal of the uncaptured matter (i.e., insoluble matter, soluble matter, and other biomolecules that are not of interest) from the sample.

FIG. 5C illustrates an exploded view of an elution setup, during which an elution solution appropriate for a specific biomolecule-solid phase complex can be added to disturb the interaction between the biomolecule and the solid phase. The elution solution can be added to each of the reservoirs 102 and removed by vacuum filtration using an elution manifold 162 and elution manifold collar 164. As illustrated in FIG. 5C, the biomolecule isolation system 150 further includes a second multi-well plate 166 which can be positioned in fluid communication with the first multi-well plate 105 to receive the biomolecule 122 (and any solvent) after being eluted from the solid phase 106. The second multi-well plate 166 includes a plurality of the second reservoirs 120 in fluid communication with the plurality of reservoirs 102 in the first multi-well plate 105. The second reservoirs 120 are positioned to receive the isolated biomolecule 122 as describe above and illustrated in FIG. 3. As is well-known to those of ordinary skill in the art, the biomolecule 122 can then be isolated from the elution solution by a variety of known techniques, including, without limitation, chromatography fractionation in a chromatography column, dialysis, centrifugation, gravity filtration, vacuum filtration, etc., and a combination thereof.

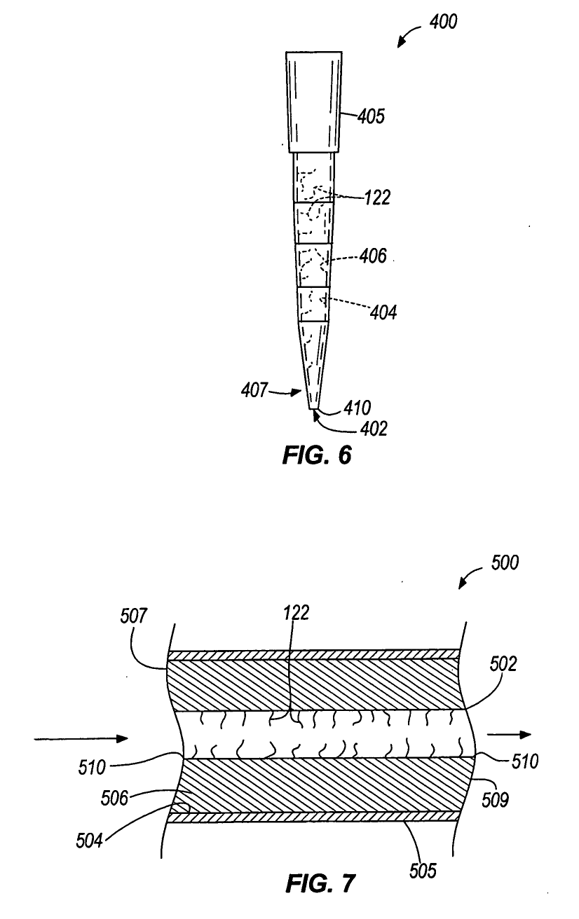

The biomolecule isolation system 150 illustrated in FIGS. 5A-5C is described above with reference to the biomolecule isolation apparatus 100 and is described as including a plurality of the biomolecule isolation apparatuses 100. However, in some embodiments, the biomolecule isolation system 150 includes a plurality of the biomolecule isolation apparatuses 200, as illustrated in FIG. 4 and described above. In addition, the biomolecule isolation system 150 can include a plurality of biomolecule isolation apparatuses 400, a plurality of biomolecule isolation apparatuses 500, a plurality of biomolecule isolation apparatuses 600, a plurality of biomolecule isolation apparatuses 700, and/or a plurality of biomolecule isolation apparatuses 800, illustrated in FIGS. 6-10, respectively, and described below. In some embodiments, the biomolecule isolation system 150 includes at least one of the biomolecule isolation apparatus 100, the biomolecule isolation apparatus 200, the biomolecule isolation apparatus 400, the biomolecule isolation apparatus 500, the biomolecule isolation apparatus 600, the biomolecule isolation apparatus 700, the biomolecule isolation apparatus 800, and combinations thereof.

FIG. 6 illustrates a biomolecule isolation apparatus 400 according to another embodiment of the invention. The biomolecule isolation apparatus 400 includes a reservoir 402 defined by a pipette tip 405. The reservoir 402 includes an inner surface 404. At least a portion of the inner surface 404 includes a solid phase 406 adapted to capture a biomolecule 122 of interest from the sample. The portion of the inner surface 404 that includes the solid phase 406 can be textured, similar to the textured inner surface 204 illustrated in FIG. 4, such that the portion of the inner surface 404 that includes the solid phase 406 has an increased surface area. The portion of the inner surface 404 that acts as the solid phase 406 in the biomolecule isolation apparatus 400 can be formed of a material that inherently captures a biomolecule 122 of interest from a sample, or the inner surface 404 can be charged, coated or otherwise modified to capture the biomolecule 122 of interest.

A sample containing the biomolecule 122 of interest can be added to the reservoir 402 and combined with the solid phase 406 using standard pipetting procedures known to those having ordinary skill in the art. For example, the sample can be drawn into an aperture 410 defined in a tip portion 407 of the pipette tip 405 to fill at least a portion of the volume of the reservoir 402 defined by the interior of the pipette tip 405. The sample can then be held, swished and/or shaken within the reservoir to allow the biomolecule 122 to interact with the solid phase 406. After a sufficient amount of time has passed to allow the biomolecule 122 to interact with the solid phase 406, the insoluble matter and any uncaptured matter can be removed from the reservoir 402 by expelling the matter from the reservoir 402 using standard pipetting procedures. The biomolecule 122 can then be removed from the solid phase 406 using any of the removal techniques described above. For example, a wash solution can be drawn into the aperture 410 defined in the tip portion 407 of the pipette tip 405 to enhance removal of uncaptured matter from at least one of the sample, the solid phase 406, and the reservoir 402. In addition, an elution solution can be drawn into the pipette tip 405 in a similar manner to disturb the interaction between the biomolecule 122 and the solid phase 406. The elution solution can be expelled using standard pipetting procedures, and the isolated biomolecule 122 of interest can be collected. The isolated biomolecule 122 of interest can be collected in a second reservoir (not shown) positioned in fluid communication with the aperture 410. In addition, repeated elution steps and washing steps can also be performed using similar techniques.

FIG. 7 illustrates a biomolecule isolation apparatus 500 according to another embodiment of the invention. The biomolecule isolation apparatus 500 includes a reservoir 502 defined by a capillary column 505. The reservoir 502 includes an inner surface 504. At least a portion of the inner surface 504 includes a solid phase 506 adapted to capture a biomolecule 122 of interest from the sample. The inner surface 504 can include the solid phase 506 by being formed of a material that inherently captures the biomolecule 122 of interest, or the inner surface 504 can be charged, coated or otherwise modified to capture the biomolecule 122 of interest.

In the embodiment illustrated in FIG. 7, the inner surface 504 is coated with the solid phase 506. In some embodiments, however, the material that forms the inner surface 504 also functions as the solid phase 506 itself. In such embodiments, the inner surface 504 can be textured, similar to the textured inner surface 204 illustrated in FIG. 4, such that the portion of the inner surface 504 that includes the solid phase 506 has an increased surface area.

A sample containing the biomolecule 122 of interest can be added to the reservoir 502 and combined with the solid phase 506 by flowing the sample through the capillary column 505 using systems and techniques known to those having ordinary skill in the art. For example, the sample can be introduced through an aperture 510 defined by an inlet portion 507 of the capillary column 505 and moved through the reservoir 502 (as shown by the arrows in FIG. 7) and out an aperture 510 defined by an outlet portion 709. The sample can be moved through the reservoir 502 at a predetermined flow rate to allow the biomolecule 122 of interest in the sample to interact with the solid phase 506. The sample can flow through the reservoir 502 at a uniform rate, or the flow rate can be altered. For example, the flow rate of the sample can be decreased or stopped to allow sufficient interaction between the biomolecule 122 and the sample, and the flow rate can be increased to enhance removal of uncaptured matter from the sample and reservoir 502. In addition, the capillary column 505 can include several sections along its length that include the solid phase 506. As illustrated in FIG. 7, the capillary column 505 can have any length desired, and the solid phase 506 can be present in a portion of the length, or the solid phase 506 can be present throughout the length of the capillary column 505.

The insoluble matter, and any other uncaptured matter, in the sample can be removed from the reservoir 502 by continuing to move the sample through the reservoir 502 using standard capillary column systems and procedures. After the insoluble matter, and any other uncaptured matter, has been removed from the reservoir 502, a wash solution can be moved through the reservoir 502 to enhance removal of uncaptured matter from at least one of the sample, the solid phase 506, and the reservoir 502. Following the wash solution, an elution solution can be moved through the reservoir 502 to disturb the interaction between the biomolecule 122 and the solid phase 506. The isolated biomolecule 122 of interest can be collected in a second reservoir (not shown) positioned in fluid communication with the aperture 510 defined by the outlet portion 509. In addition, repeated elution steps and washing steps can also be performed using similar techniques.

FIG. 8 illustrates a biomolecule isolation apparatus 600 according to another embodiment of the present invention, wherein like numerals represent like elements. The biomolecule isolation apparatus 600 shares many of the same elements and features described above with reference to the biomolecule isolation apparatus 100 of FIGS. 1-3, except that the biomolecule isolation apparatus 600 includes a reservoir 602 that is defined by a pipette tip 605 (similar to the pipette tip 405 illustrated in FIG. 6 and described above). Accordingly, elements and features corresponding to elements and features in the embodiment illustrated in FIGS. 1-3 are provided with the same reference numerals in the 600 series. Reference is made to the description above accompanying FIGS. 1-3 for a more complete description of the features and elements (and alternatives to such features and elements) of the embodiment illustrated in FIGS. 1-3.

As illustrated in FIG. 8, the biomolecule isolation apparatus 600 includes a reservoir 602 having an inner surface 604, a solid phase 606 that includes a plurality of particles 616 contained within the reservoir 602 and adapted to capture a biomolecule 122 from a sample, a filter 608 positioned between the solid phase 606 and at least a portion of the inner surface 604, a seal-forming device 612 (e.g., an o-ring) positioned adjacent the periphery of the filter 608 and a portion of the inner surface 604 to maintain an adequate seal around the periphery of the filter 608, and an aperture 610 defined in the inner surface 604, and particularly, defined in a tip portion 607 of the pipette tip 605.

The filter 608 allows matter from the sample that has not been captured by the solid phase 606 to be removed from the reservoir 602 from the tip portion 607, while maintaining the solid phase 606, along with the biomolecule 122 that has been captured, within the reservoir 602. The filter 608 can include any of the types of filters mentioned above, and combinations thereof.

In some embodiments, the biomolecule isolation apparatus 600 does not include the filter 608. For example, in some embodiments, the solid phase 606 includes one or more relatively large particles 616. In some embodiments, the particles 616 are sized such that the particles 616 will be retained in the reservoir 602 without the use of the filter 608. In such embodiments, the size of the particles 616 can be at least partially dependent on the width and the degree of taper of the tip portion 607 of the pipette tip 605. Furthermore, one or more apertures 610 can be defined in the inner surface 604 of the reservoir 602 to allow insoluble matter to pass out of the reservoir 602 while retaining the solid phase 606 within the reservoir 602.

A sample containing the biomolecule 122 of interest can be added to the reservoir 602 and combined with the solid phase 606 using standard pipetting procedures. For example, the sample can be drawn into the aperture 610 defined in the tip portion 607 of the pipette tip 605 to fill at least a portion of the volume of the reservoir 602 defined by the interior of the pipette tip 605. The sample can then be held, swished, and/or shaken within the reservoir to allow the biomolecule 122 to interact with the solid phase 606. After a sufficient amount of time has passed to allow the biomolecule 122 to interact with the solid phase 606, the insoluble matter and any other uncaptured matter in the sample can be removed from the reservoir 602 by expelling the sample from the tip portion 607 of the pipette tip 605 using standard pipetting procedures. The biomolecule 122 can then be removed from the solid phase 606 using any of the removal techniques described above. For example, a wash solution can be drawn into the aperture 610 defined in the tip portion 607 of the pipette tip 605 to remove uncaptured matter from the reservoir 602. In addition, an elution solution can be drawn into the pipette tip 605 in a similar manner to disturb the interaction between the biomolecule 122 and the solid phase 606. The elution solution can be expelled using standard pipetting procedures, and the isolated biomolecule 122 of interest can be collected. The isolated biomolecule 122 of interest can be collected in a second reservoir (not shown) positioned in fluid communication with the aperture 610. In addition, repeated elution steps and washing steps can also be performed using similar techniques.

FIG. 9 illustrates a biomolecule isolation apparatus 700 according to another embodiment of the present invention, wherein like numerals represent like elements. The biomolecule isolation apparatus 700 shares many of the same elements and features described above with reference to the biomolecule isolation apparatus 100 of FIGS. 1-3, except that the biomolecule isolation apparatus 700 includes a reservoir 702 that is defined by a capillary column 705 (similar to the capillary column 505 illustrated in FIG. 7 and described above). Accordingly, elements and features corresponding to elements and features in the embodiment illustrated in FIGS. 1-3 are provided with the same reference numerals in the 700 series. Reference is made to the description above accompanying FIGS. 1-3 for a more complete description of the features and elements (and alternatives to such features and elements) of the embodiment illustrated in FIGS. 1-3.

As illustrated in FIG. 9, the biomolecule isolation apparatus 700 includes a reservoir 702 having an inner surface 704, a solid phase 706 that includes a plurality of particles 716 contained within the reservoir 702 and adapted to capture a biomolecule 122 from a sample, two filters 708 positioned between the solid phase 706 and at least a portion of the inner surface 704, a seal-forming device 712 (e.g., an o-ring) positioned adjacent the periphery of each filter 708 and a portion of the inner surface 704 to maintain an adequate seal around the periphery of each filter 708, and two apertures 710 defined in the inner surface 704, and particularly, defined by an inlet portion 707 and an outlet portion 709 of the capillary column 705.

A sample containing the biomolecule 122 of interest can be added to the reservoir 702 and combined with the solid phase 706 by flowing the sample through the capillary column 705 using systems and techniques known to those having ordinary skill in the art. For example, the sample can be introduced through an aperture 710 defined by the inlet portion 707 of the capillary column 705 and moved through the reservoir 702 (as shown by the arrows in FIG. 9) and out an aperture 710 defined by the outlet portion 709. The sample can be moved through the reservoir 702 at a predetermined flow rate to allow the biomolecule 122 of interest in the sample to interact with the solid phase 706. The sample can flow through the reservoir 702 at a uniform rate, or the flow rate can be altered. For example, the flow rate of the sample can be decreased or stopped to allow sufficient interaction between the biomolecule 122 and the sample, and the flow rate can be increased to enhance removal of any uncaptured matter from the sample and reservoir 702.

As illustrated in FIG. 9, the capillary column 705 can have any length desired, and the distance between the two filters 708 can be varied. In addition, the capillary column 705 can include several sections along its length that include the solid phase 706. As a result, the solid phase 706 can be present in a portion of the length of the capillary column 705, or the solid phase 706 can be present throughout the length of the capillary column 705.

The insoluble matter and any uncaptured matter in the sample can be removed from the reservoir 702 by continuing to move the sample through the reservoir 702 using standard capillary column systems and procedures. After the insoluble and any uncaptured matter has been removed from the reservoir 702, a wash solution can be moved through the reservoir 702 to more completely remove uncaptured matter from the sample and the solid phase 706. Following the wash solution, an elution solution can be moved through the reservoir 702 to disturb the interaction between the biomolecule 122 and the solid phase 706. The isolated biomolecule 122 of interest can be collected a second reservoir (not shown) positioned in fluid communication with the aperture 710 defined by the outlet portion 709.

In some embodiments, the biomolecule isolation apparatus 700 does not include one or both of the two filters 708. For example, in some embodiments, only one filter 708 is used, because the flow of the sample through the reservoir 702 maintains the particles 716 of the solid phase 706 in position to capture the biomolecule 122 of interest. That is, in some embodiments, the filter 108 on the left side of FIG. 9 is omitted. In addition, in some embodiments, the capillary column is shaped such that the particles 716 will be retained in the reservoir 702 without the filters 708. Furthermore, one or more apertures 710 can be defined in the inner surface 704 of the reservoir 702 to allow insoluble matter to pass out of the reservoir 702 while retaining the solid phase 706 within the reservoir 702. In addition, repeated elution steps and washing steps can also be performed using similar techniques.

FIG. 10 illustrates a biomolecule isolation apparatus 800 according to another embodiment of the present invention, wherein like numerals represent like elements. The biomolecule isolation apparatus 800 shares many of the same elements and features described above with reference to the biomolecule isolation apparatus 100 of FIGS. 1-3, except that the biomolecule isolation apparatus 800 includes a reservoir 802 that is defined by a basket 805. Accordingly, elements and features corresponding to elements and features in the embodiment illustrated in FIGS. 1-3 are provided with the same reference numerals in the 800 series. Reference is made to the description above accompanying FIGS. 1-3 for a more complete description of the features and elements (and alternatives to such features and elements) of the embodiment illustrated in FIGS. 1-3.

As illustrated in FIG. 10, the biomolecule isolation apparatus 800 includes a reservoir 802 having an inner surface 804, a solid phase 806 that includes a plurality of particles 816 contained within the reservoir 802 and adapted to capture a biomolecule 122 from a sample, and a filter 808 defined at least partially by the reservoir 802 of the basket 805.

A sample containing the biomolecule 122 of interest can be added to the reservoir 802 and combined with the solid phase 806 by dipping at least a portion of the basket 805 into a container that contains the sample. As the basket 805 is dipped into the sample, the sample is allowed to flow through pores 811 of the filter 808, and into the reservoir 802 where the sample can interact with the solid phase 806. In this embodiment, the interaction of the sample and the solid phase 806 is not dependent on flow rate through the reservoir 802, but rather is at least partially dependent on the amount of time that the basket 805 is held in contact with the sample. To remove the uncaptured matter from at least one of the sample, the reservoir 802 and the solid phase 806, the basket 805 can be lifted out of the sample, or the uncaptured matter can be decanted or siphoned off.

The basket 805 and the solid phase 806 can then be washed by rinsing or spraying the basket 805 with a wash solution, or by dipping the basket 805 into a wash solution and then removing the basket 805 from the wash solution. Similarly, the biomolecule 122 of interest can be removed from the solid phase 806 by rinsing or spraying the basket 805 with an elution solution and collecting what comes off of the solid phase 806. The biomolecule 122 can instead be removed from the solid phase 806 by dipping the basket 805 into an elution solution and then removing the basket 805 from the elution solution. Repeated elution steps and washing steps can be performed using similar techniques.

The filter 808 illustrated in FIG. 10 is shown as being defined by sides 813 and a bottom 815 of the basket 805. However, in some embodiments, the filter 808 is defined by a portion of the sides 813 and/or a portion of the bottom 815 of the basket 805. In some embodiments, the biomolecule isolation apparatus 800 does not necessarily include the filter 808, but rather includes one or more apertures defined in the inner surface 804 of the reservoir 802 to allow insoluble matter to pass out of the reservoir 802 while retaining the solid phase 806 within the reservoir 802.

The embodiment illustrated in FIG. 10 shows a schematic example of how a biomolecule isolation apparatus can include a basket-defined reservoir 802. Accordingly, the basket 805 includes a handle 817, which can be gripped by a user or an automatic device. However, it should be noted that the basket 805 can be one of a plurality of baskets 805 (similar to a plurality of wells in a multi-well plate) that are dipped into a plurality of samples, and the handle 817 need not be included.

In addition, the basket 805 illustrated in FIG. 10 has an open end 819, but it should be noted that in some embodiments, the basket 805 is closed on all sides, thereby forming a cage that can be dropped, dipped, etc. into a sample, a wash solution, and an elution solution.

Furthermore, the basket 805 illustrated in FIG. 10 is formed of a rigid material. However, it should be noted that in some embodiments, the basket 805 is formed of a soft material, including a woven cloth mesh, a woven plastic mesh, etc., and combinations thereof. In embodiments employing a soft basket 805, basket 805 can include the open end 819, or the basket 805 can be closed.

In the embodiment illustrated in FIG. 10, the solid phase 806 includes a plurality of particles 816. However, in some embodiments, the filter 808 is charged, coated or otherwise modified to include the solid 806 that is adapted to capture the biomolecule of interest 122. The modified filter 808 can be used in lieu of, or in addition to, the particles 816 to make up the solid phase 806.

A variety of combinations of any of the solid phases 106, 206, 406, 506, 606, 706, 806 can be used to isolate a biomolecule 122 from a sample without departing from the spirit and scope of the present invention, as long as the solid phase 106, 206, 406, 506, 606, 706, 806 allows the insoluble matter of the sample to flow through or out of the biomolecule isolation apparatus 100, 200, 400, 500, 600, 700, 800 without substantially clogging.

In any of the biomolecule isolation apparatuses 100, 200, 400, 500, 600, 700, 800 described above, one or more solid phases 106, 206, 406, 506, 606, 706, 806 can be used to isolate one or more biomolecules 122 from a sample. Wash solutions and elution solutions can be chosen to selectively wash and remove the biomolecules 122 from the solid phases 106, 206, 406, 506, 606, 706, 806.

As mentioned above, existing systems for isolating a biomolecule 122 require initial removal of any insoluble matter from the sample before the sample can be combined with any solid phase. However, the present invention allows the sample, including soluble and insoluble matter, to be added directly to the solid phase, and the insoluble matter to be removed from the sample after the solid phase has been combined with the sample. As a result, removing the insoluble matter from the sample occurs after combining the solid phase with the sample of the present invention. In addition, in the present invention, the solid phase can be combined with the sample without any prior filtration, separation or purification of the sample.

A variety of biomolecules 122 can be isolated from the sample of complex biological materials, including, without limitation, the biomolecules 122 listed in Table 1. Accordingly, a variety of solid phases 106 can be used to isolate the various biomolecules 122 from a sample, which are also listed in Table 1. In some embodiments, the solid phase 106 includes at least one of silica, agarose, sepharose, acrylamide, latex, etc., and combinations thereof, which can inherently capture a variety of biomolecules 122, or which can be modified to capture a variety of biomolecules 122.

Specifically, as shown in Table 1, sequence-specific nucleic acids can be isolated from a sample using a sequence-specific nucleic acid solid phase; his tagged proteins can be isolated using a metal-charged solid phase (e.g., one of the solid phases listed above can be charged with nickel, zinc, and combinations thereof; HISLINK™ purification product available from Promega Corporation, Madison, Wis., catalog no. V8821); biotinylated biomolecules can be isolated using a solid phase comprising streptavidin; mRNA can be isolated from a sample using oligo dT associated with, complexed with, or bound to a solid phase; total RNA can be isolated using a silica solid phase; genomic DNA can be isolated using a silica solid phase (see Example 2); plasmid DNA can be isolated using a silica solid phase or a metal-charged solid phase; plant DNA can be isolated using a silica solid phase or a metal-charged solid phase; fractionation of proteins from a sample can be accomplished using an anion exchange resin (e.g., a solid phase that includes a trimethylbenzylammonium group as an exchange site); fractionation of proteins from a sample can be accomplished using a cation exchange resin (e.g., a solid phase that includes sulfonic acid as an exchange site); fractionation of proteins from a sample can be accomplished using a size exclusion chromatography resin; glutathione-S-transferase (GST) fusion proteins can be isolated using a glutathione solid phase; and an immunoassay (e.g., ELISA) can be performed using a solid phase that comprises the corresponding antibody or antigen.

TABLE 1
Biomolecules of interest and corresponding solid phases that can
be used to isolate the biomolecules.

| Biomolecule of Interest | Solid Phase |
|---|---|
| Purification of sequence- | Sequence-specific nucleic acid solid phase |
| specific nucleic acids | |
| Purification of his tagged | Metal-charged solid phase |
| protein | |
| Purification of thioredoxin | Phenylarsine oxide-modified (ThioBond) |
| tagged protein | resin |
| Purification of biotinylated | Streptavidin solid phase |
| biomolecule | |
| Purification of mRNA | Oligo dT solid phase |
| Purification of total RNA | Silica solid phase |
| Purification of genomic DNA | Silica solid phase |
| Purification of plasmid DNA | Silica solid phase or metal-charged solid |
| | phase |
| Purification of plant DNA | Silica solid phase or metal-charged solid |
| | phase |
| Fractionation of proteins | Anion exchange resin |
| Fractionation of proteins | Cation exchange resin |
| Fractionation of proteins | Size exclusion chromatography resin |
| Purification of GST fusion | Glutathione solid phase |
| proteins | |
| Immunoassay (ELISA) | Antibody/Antigen solid phase |

Other biomolecules and corresponding solid phases can be used without departing from the spirit and skill of the present invention. One of ordinary skill in the art can select a solid phase, or modify an existing solid phase to isolate a biomolecule 122 of interest from a sample using a variety of bioaffinity tags. The bioaffinity tags can include, without limitation, antibodies, DNA probes, RNA probes, positively charged groups, negatively charged groups, etc., and combinations thereof.

By way of example only, mRNA can be isolated from a sample in a variety of ways. In some embodiments, a biotinylated oligo dT probe can be attached to any of the solid phases 106, 206, 406, 506, 606, 706, 806 via a steptavidin interaction (using a variety of techniques known to those of ordinary skill in the art). Then, the poly(A) tails of the mRNA in the sample can hybridize with the oligo dT probe as the sample flows past the solid phase 106, 206, 406, 506, 606, 706, 806.

In some embodiments, streptavidin can be attached to any of the solid phases 106, 206, 406, 506, 606, 706, 806 (using a variety of techniques known to those of ordinary skill in the art). In addition, a biotinylated oligo dT probe can be hybridized to the poly(A) tails of the mRNA in the sample. In such embodiments, the biotin-streptavidin interaction between the biotinylated-mRNA in the sample and the solid phase 106, 206, 406, 506, 606, 706, 806 modified with streptavidin isolates the mRNA from the sample. In the embodiments in which the solid phase 106, 206, 406, 506, 606, 706, 806 is modified with streptavidin, the solid phase 106, 206, 406, 506, 606, 706, 806 can be used to isolate a variety of biomolecules 122 without having to manufacture entirely new and different solid phases 106, 206, 406, 506, 606, 706, 806 for each biomolecule 122 of interest. However, both of the methods described above can be used without departing from the spirit and scope of the present invention, and similar alternatives exist for each biomolecule 122 desired to be isolated. One of ordinary skill in the art will recognize how to alter the biomolecule isolation system (such as the biomolecule isolation system 150 described above and illustrated in FIGS. 5A-5C) and method for each biomolecule 122 of interest.

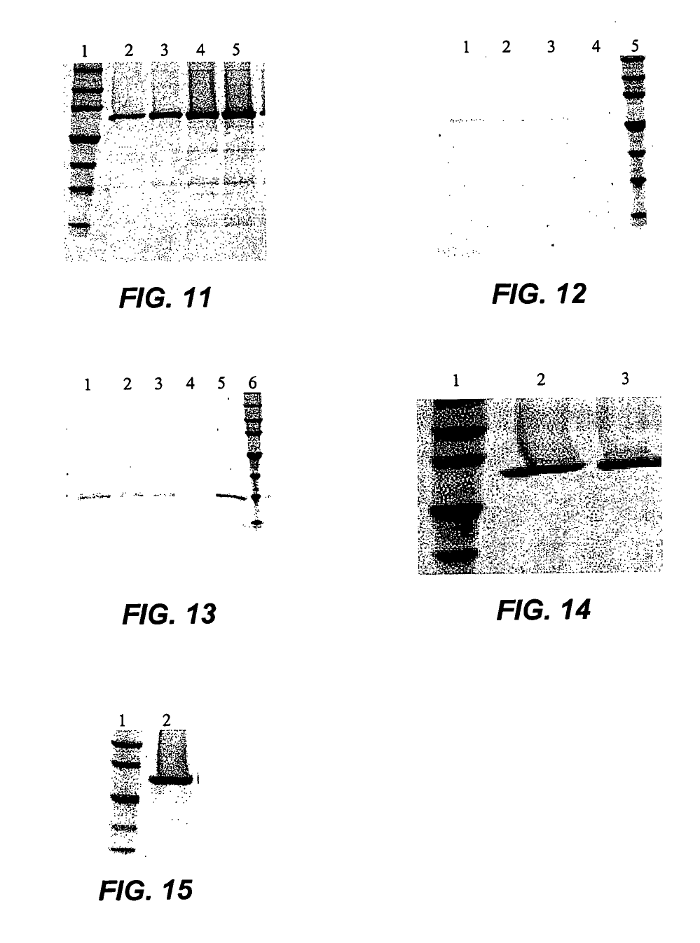

Working Examples 1-7 relate generally to biomolecule isolation apparatuses, systems and methods, and FIGS. 11-15 correspond to Examples 4-7.

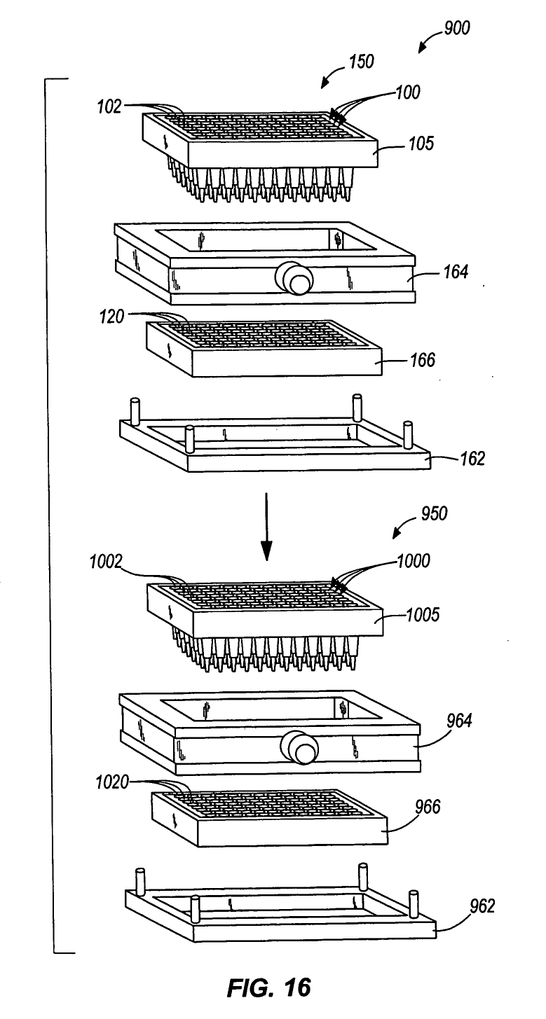

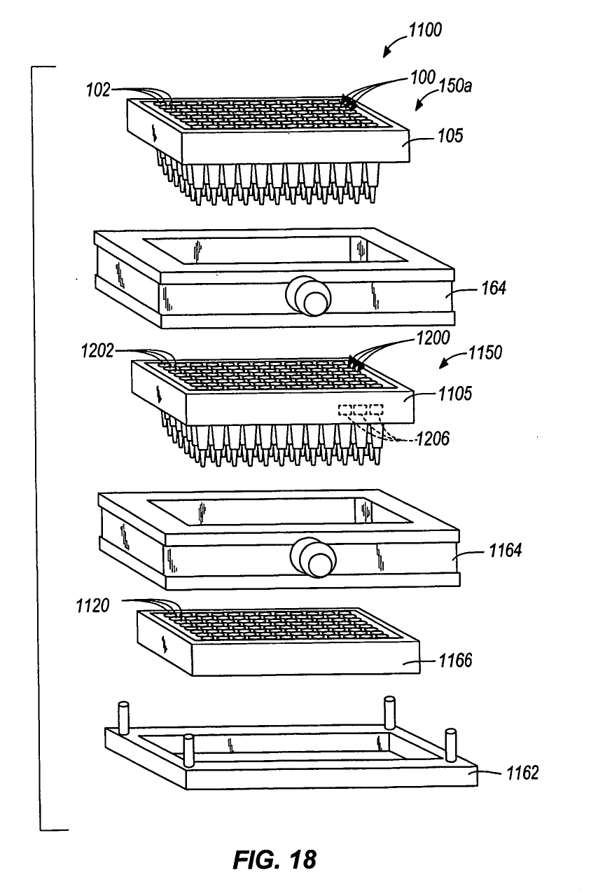

FIG. 16 illustrates a contaminant removal system 900 and method according to one embodiment of the present invention. FIG. 18 illustrates a contaminant removal system 1100 and method according to another embodiment of the present invention. In some embodiments of the present invention, an isolated biomolecule 122 of interest can include or be associated with one or more contaminants after being removed from the solid phase 106, 206, 406, 506, 606, 706, 806 of the biomolecule isolation apparatus 100, 200, 400, 500, 600, 700, 800, as described in greater detail below. The presence of the contaminants can affect in vitro and/or in vivo downstream processes or applications, including, without limitation, functional assays, interaction analysis, quantitation, structural analysis, mass spectrometry measurements, NMR measurements, crystallization trials, and combinations thereof. Examples 8-15 describe several examples of samples that include biomolecules of interest and one or more contaminants that can be separated using a contaminant removal system 900, 1100 of the present invention.

The contaminant removal system 900 includes the elution setup portion of the biomolecule isolation system 150, as described above with respect to FIG. 5C, and a fractionation system 950. The biomolecule isolation system 150 includes the same elements and features described above with reference to the illustrated embodiment of FIGS. 5A-5C. Reference is made to the description above accompanying FIGS. 5A-5C for a more complete description of the features and elements (and alternatives to such features and elements) of the embodiment illustrated in FIG. 16.

The biomolecule isolation system 150 is illustrated in FIG. 16 as part of the contaminant removal system 900 by way of example only. However, the contaminant removal system 900 can include a variety of biomolecule isolation systems, including systems that incorporate any biomolecule isolation apparatus 100, 200, 400, 500, 600, 700, 800 of the present invention, other filtration techniques or systems not specifically discussed herein, and any other isolation system capable of isolating a biomolecule 122 of interest from a sample. In some embodiments, the fractionation system 950 is used separately and independently from any biomolecule isolation system to separate a biomolecule 122 of interest from one or more contaminants.

With continued reference to the embodiment illustrated in FIG. 16, the first reservoirs 102 of the first multi-well plate 105 include the isolated biomolecule 122 of interest captured by the solid phase 106 after undesirable matter (including soluble matter, other biomolecules not of interest, and any insoluble matter) has already been removed from the sample. Accordingly, the biomolecule 122 of interest is ready to be removed from the solid phase 106 by various methods, including elution. For example, as explained above with reference to FIG. 5C, an elution solution appropriate for a specific biomolecule-solid phase complex can be added to disturb the interaction between the biomolecule and the solid phase 106. The elution solution can be added to each of the reservoirs 102 and removed by vacuum filtration using the elution manifold 162 and the elution manifold collar 164.

Accordingly, after the biomolecule 122 of interest has been eluted from the solid phase 106, the second reservoirs 120 include an eluate that includes the biomolecule 122 of interest and one or more contaminants (i.e., free molecules or molecules bound to the biomolecule 122) including, without limitation, at least one of an elution molecule, contaminating debris or portions of the solid phase 106 that have leached through the filter 108 in the biomolecule isolation apparatus 100, metals, dye molecules, label molecules, salts, endotoxins, etc.

The eluate in the second reservoirs 120 can then be transferred to the fractionation system 950, and specifically to a fractionation multi-well plate 1005 in the fractionation system 950. The fractionation system 950 shares many of the same elements and features described above with reference to the biomolecule isolation system 150 of FIGS. 5C and 16. Accordingly, the elements and features of the fractionation system 950 that correspond to elements and features of the biomolecule isolation system 150 are provided with the same reference numerals in the 900 series. Reference is made to the description above accompanying FIGS. 5C for a more complete description of the features and elements (and alternatives to such features and elements) of the fractionation system 950 of FIG. 16.

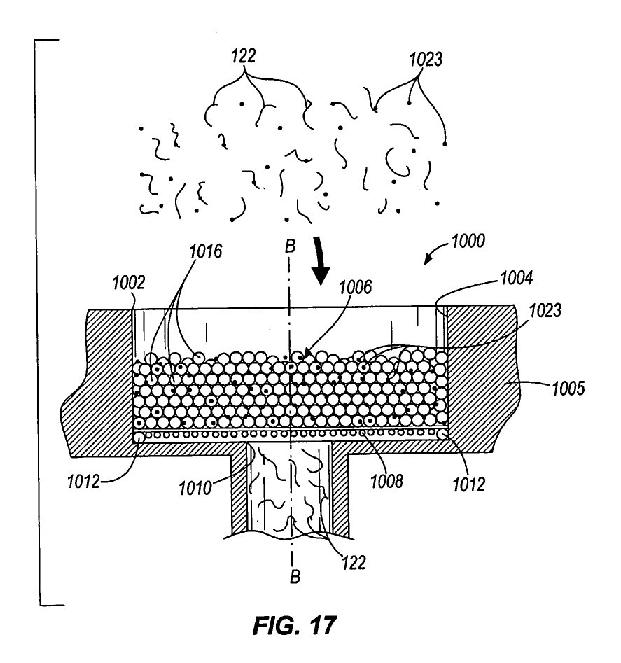

As discussed above, the fractionation multi-well plate 1005 includes a plurality of fractionation devices 1000. The fractionation multi-well plate 1005 is an example of a unitary device that includes a plurality of fractionation devices 1000. That is, the plurality of fractionation devices 1000 of the present invention are part of a larger, unitary device that can be used to separate the biomolecule 122 of interest from one or more contaminants in a high volume and/or high-throughput manner. One embodiment of the fractionation device 1000 is shown in cross-section in FIG. 17. FIG. 17 illustrates one embodiment of a fractionation device 1000 according to the present invention. The fractionation device 1000 shares many of the same elements and features described above with reference to the biomolecule isolation apparatus 100 of FIGS. 1-3. Accordingly, elements and features corresponding to elements and features in the biomolecule isolation apparatus 100 are provided with the same reference numerals in the 1000 series. Reference is made to the description above accompanying FIGS. 1-3 for a more complete description of the features and elements (and alternatives to such features and elements) of the fractionation device 1000 illustrated in FIG. 17.

Thus, it should be understood that any of the alternative structures described above for the biomolecule isolation apparatus of the present invention, including a reservoir/well of a multi-well plate, a capillary column, a pipette tip, a basket, etc., can also be used for a fractionation device of the present invention. Accordingly, the unitary device can include any unitary structure having a plurality of fractionation devices, including, without limitation, at least one of a multi-well plate, a structure comprising a plurality of capillary columns, a structure comprising a plurality of pipette tips, a structure comprising a plurality of baskets, etc., and combinations thereof. For example, the unitary device can include a structure that resembles a multi-well plate that includes a plurality of baskets instead of a plurality of reservoirs/wells, or the unitary device can include a multi-pipette tip pipetting device, wherein each pipette tip is a fractionation device of the present invention. Other structures for the unitary device are possible and within the spirit and scope of the present invention.

With reference to FIG. 17, the fractionation device 1000 includes a reservoir 1002 having an inner surface 1004 and a longitudinal axis B-B, a solid phase 1006 contained within the reservoir 1002, a filter 1008 positioned between the solid phase 1006 and at least a portion of the inner surface 1004, a seal-forming device 1012 (e.g., an o-ring) positioned adjacent the periphery of the filter 1008 and a portion of the inner surface 1004 to maintain an adequate seal around the periphery of the filter 1008, and an aperture 1010 defined in the inner surface 1004 of the reservoir 1002.

The filter 1008 is adapted to allow the sample (e.g., the eluate transferred from the second reservoirs 120 of the biomolecule isolation system 150) to pass therethrough, while substantially preventing the solid phase 1006 from passing therethrough. The solid phase 1006 includes a fractionation chromatography medium that is adapted to separate the biomolecule 122 of interest from one or more contaminants 1023 that may be present in the sample. In some embodiments, the fractionation chromatography medium of the solid phase 1006 includes a gel filtration matrix. The gel filtration matrix can include small porous particles 1016, as shown in FIG. 17. The porous particles 1016 can be adapted and/or selected such that the biomolecule 122 of interest and the contaminant(s) 1023 take varying amounts of time to transit through the solid phase 1006 (and, accordingly, the fractionation reservoir 1002), thereby allowing separation of the biomolecules 122 of interest and the contaminants(s) 1023.

In some embodiments, as shown in FIG. 17, the biomolecule 122 of interest is a larger molecule than the contaminant(s) 1023. In the embodiment illustrated in FIG. 17, the gel filtration porous particles 1016 are adapted to allow the biomolecule 122 of interest to transit through the solid phase 1006 at a faster rate than the contaminant(s) 1023. That is, the porous particles 1016 are sized such that the biomolecule 122 of interest is too large to become caught in the pores of the porous particles 1016, and transits through the column more rapidly than the contaminant(s) 1023 by moving around and past the porous particles 1016. That is, the biomolecule 122 of interest is “excluded” from the gel filtration matrix of the solid phase 1006. The smaller contaminant(s) 1023, however, can enter the pores of the porous particles 1016 and transit through the porous particles 1016, thereby taking a longer amount of time to transit through the solid phase 1006 and the fractionation reservoir 1002. As a result, the fractionation device 1000 illustrated in FIGS. 16 and 17 is a type of size exclusion fractionation device. This type of fractionation device is also sometimes referred to as a molecular sieve fractionation device in that the components of a sample can be separated according to their molecular size (and to a certain extent, molecular shape).

In some embodiments, the gel filtration matrix of the solid phase 1006 can be formed of at least one of crosslinked polysaccharides and crosslinked polyacrylamide, each of which can include porous particles 1016 of varying pore sizes. A large variety of samples including a biomolecule 122 of interest and one or more contaminants 1023 can be separated by using gel filtration matrices having porous particles 1016 of varying sizes and porosities. Examples of solid phases 1006 that can be used in size exclusion fractionation devices can include, without limitation, at least one of at least one of SEPHADEX™ G10-200 separation particles (available from Amersham), SEPHACRYL™ S-100-S-1000 separation particles (available from Amersham), SEPHAROSE™ 2B-6B separation particles (available from Amersham), BIO-GEL™ A-0.5 m-150 m separation particles (available from Bio-Rad), and combinations thereof.

The size exclusion fractionation device 1000 is explained and illustrated by way of example only. However, the fractionation system 950 can include a variety of other types of fractionation devices, including, without limitation, ion exchange fractionation devices and affinity fractionation devices. These other types of fractionation devices function in a similar manner to separate a biomolecule of interest from one or more contaminants by affecting the relative amount of time it takes for the biomolecule and contaminants to transit through the device.

With continued reference to FIG. 16, after the sample comprising the biomolecule 122 of interest and contaminant(s) 1023 is transferred to the reservoirs 1002 of the fractionation multi-well plate 1005, the sample can be moved through the solid phase 1006 into a collection plate 966 by a variety of methods, including gravity filtration, vacuum filtration (as shown in FIG. 16), centrifugation, etc., and combinations thereof. The collection plate 966 includes a plurality of collection reservoirs 1020, and each collection reservoir 1020 is in fluid communication with a fractionation reservoir 1002 of the fractionation multi-well plate 1005. The collection reservoirs 1020 of the collection plate 966 can be used to collect the biomolecule 122 of interest.

As described above with reference to FIG. 17, the illustrated embodiment of the fractionation device 1000 is a size exclusion fractionation device adapted to increase the time it takes for the contaminant(s) 1023 to transit through the fractionation reservoir 1002 relative to the biomolecule 122 of interest. Accordingly, the collection plate 966 can be disconnected from the fractionation multi-well plate 1005 after an appropriate amount of time has passed to allow the biomolecule 122 of interest to pass through the fractionation reservoir 1002, without allowing the contaminant(s) 1023 to pass through the fractionation reservoir 1002. Alternatively, in a fractionation device (size exclusion or otherwise) adapted to allow the contaminant(s) 1023 to pass through the fractionation reservoir 1002 first, the contaminant(s) 1023 can be collected in a first collection plate (not shown) or disposed, and subsequently, the collection plate 966 can be fluidly coupled to the fractionation multi-well plate 1005 to collect the biomolecule 122 of interest at an appropriate point in time.

FIG. 18 illustrates a contaminant removal system 1100 and method according to another embodiment of the present invention, wherein like numerals represent like elements. The contaminant removal system 1100 shares many of the same elements and features described above with reference to the contaminant removal system 900 shown in FIG. 16. Accordingly, elements and features corresponding to elements and features in the contaminant removal system 900 of FIG. 16 are provided with the same reference numerals in the 1100 series. Reference is made to the description above accompanying FIG. 16 for a more complete description of the features and elements (and alternatives to such features and elements) of the contaminant removal system 1100 of FIG. 18 that are similar to those of the contaminant removal system 900 of FIG. 16.

As shown in FIG. 18, the contaminant removal system 1100 includes a partial biomolecule isolation system, which is referred to in FIG. 18 as 150a, and specifically, includes a portion of the elution setup of the biomolecule isolation system 150 shown in FIGS. 5C and 16. The partial biomolecule isolation system 150a includes the first multi-well plate 105 and the elution manifold collar 164, but does not include a multi-well plate dedicated to collecting the eluate from the first multi-well plate 105 (e.g., the second multi-well plate 166 of FIGS. 5C and 16), or an elution manifold (e.g., the elution manifold 162 of FIGS. 5C and 16).

The contaminant removal system 1100 further includes a fractionation system 1150. The fractionation system 1150 shares many of the same elements and features described above with reference to the fractionation system 950 of FIG. 16. Accordingly, elements and features corresponding to elements and features in the fractionation system 950 of FIG. 16 are provided with the same reference numerals in the 1150 series. Reference is made to the description above accompanying FIG. 16 for a more complete description of the features and elements (and alternatives to such features and elements) of the fractionation system 1150 of FIG. 18.

The partial biomolecule isolation system 150a is illustrated in FIG. 18 as part of the contaminant removal system 1100 by way of example only. However, the contaminant removal system 1100 can include a variety of biomolecule isolation systems, including systems that incorporate any biomolecule isolation apparatus 100, 200, 400, 500, 600, 700, 800 of the present invention, other filtration techniques or systems not specifically discussed herein, and any other isolation system capable of isolating a biomolecule 122 of interest from a sample. In some embodiments, the fractionation system 1150 is used separately and independently from any biomolecule isolation system to separate a biomolecule 122 of interest from one or more contaminants.

The fractionation system 1150 includes a fractionation multi-well plate 1105 that includes a plurality of fractionation devices 1200. The fractionation device 1200 is not shown in detail, but it should be understood that the fractionation device 1200 shares many of the same elements and features described above with reference to the fractionation device 1000 of FIG. 17. Accordingly, elements and features corresponding to elements and features in the fractionation device 1000 of FIG. 17 are provided with the same reference numerals in the 1200 series (but only the fractionation reservoirs 1202 and a few solid phases 1206 are shown in FIG. 18). Reference is made to the description above accompanying FIG. 17 for a more complete description of the features and elements (and alternatives to such features and elements) of the fractionation device 1200 of FIG. 18.

Each fractionation device 1200, and specifically, each fractionation reservoir 1202 of the fractionation multi-well plate 1105 is fluidly connected to a first reservoir 102 of the first multi-well plate 105. In addition, each fractionation reservoir 1202 of the fractionation multi-well plate 1105 is fluidly connected to a collection reservoir 1120 in a collection plate 1166. The collection plate 1166 fits adjacent a collection manifold 1162, and the elution manifold collar 164 and a collection manifold collar 1164 to allow the first multi-well plate 105, the fractionation multi-well plate 1105 and the collection plate 1166 to be sealingly engaged and in fluid communication. Accordingly, an elution solution appropriate for a specific biomolecule-solid phase complex can be added each first reservoir 102 of the first multi-well plate 105 to disturb the interaction between the biomolecule 122 of interest and the solid phase 106. The elution solution can be added to each of the reservoirs 102 and removed from each first reservoir 102, moved through each fractionation reservoir 1202, and into each collection reservoir 1120 by vacuum filtration using the collection manifold 1162, the elution manifold collar 164, and the collection manifold collar 1164.

Accordingly, after the biomolecule 122 of interest has been eluted from the solid phase 106, the fractionation reservoirs 1202 include an eluate that includes the biomolecule 122 of interest and one or more contaminants, such as those described above. The eluate can then be moved through the solid phase 1206 in each fractionation reservoir 1202. Only a few solid phases 1206 are shown for clarity, but it should be understood that any fractionation reservoir 1202 that is in use would also include a solid phase 1206 adapted to separate the biomolecule 122 of interest and the contaminant(s) 1023. Similar to the collection plate 966 described above, the collection plate 1166 can be used to collect the biomolecule 122 of interest after it has been separated from the contaminant(s) 1023 by fractionation using the solid phase 1206.

As shown in FIG. 18, the contaminant removal system 1100 is similar to the contaminant removal system 900 of FIG. 16, but does not require transfer of an eluate from a multi-well plate into the fractionation multi-well plate 1105. Instead, the fractionation multi-well plate 1105 is in fluid communication with the first multi-well plate 105, and the collection plate 1166. Accordingly, in the contaminant removal system 1100, the first multi-well plate 105, the fractionation multi-well plate 1105, and the collection plate 1166 are positioned in a stacked configuration.

A variety of biomolecules 122 of interest and contaminant(s) 1023 can be separated using either the contaminant removal system 900 of FIG. 16 or the contaminant removal system 1100 of FIG. 18. In some embodiments, the contaminant removal systems 900, 1100 can be used for at least one of the following applications: (1) removal an elution solution (e.g., imidazole) from an eluate (see Working Example 8 and Prophetic Examples 9, 10 and 13); (2) removal of salts (see Prophetic Example 11); (3) size exclusion removal of contaminant molecules, such as glutathione-S-transferase (GST), dyes (see Prophetic Example 12), fluorescent labels ((see Prophetic Example 12), radiolabels (e.g., ³²P; see Prophetic Example 12), unhybridized primers in a polymerase chain reaction (PCR) nucleic acid amplification process, unincorporated nucleotides in a PCR nucleic acid amplification process, “contaminant” biomolecules (e.g., proteins) that have been undesirably isolated with a biomolecule of interest, metal ions (see Prophetic Example 14), endotoxins (see Prophetic Example 15), etc., and combinations thereof.

Any of the biomolecule isolation apparatuses 100, 200, 400, 500, 600, 700, 800 of the present invention can be used with a variety of fractionation devices of the present invention in a high-volume and/or a high-throughput production system. As described above, some embodiments of the present invention include only one of either a biomolecule isolation apparatus of the present invention or a fractionation device of the present invention, while other embodiments of the present invention incorporate both a biomolecule isolation apparatus of the present invention and a fractionation device of the present invention. For example, the contaminant removal systems 900 and 1100 described above and illustrated in FIGS. 16 and 18, respectively, are illustrated by way of example only as including a biomolecule isolation system 150 (or a portion thereof) that includes a biomolecule isolation apparatus 100 of the present invention, and a fractionation system 950, 1150 that includes a fractionation device of the present invention. However, in some embodiments of the present invention, the contaminant removal system 900 or 1100 does not include any biomolecule isolation system, but rather includes only a fractionation system 950 or 1150. In such embodiments, a sample comprising a biomolecule 122 of interest and one or more contaminants 1023 (e.g., an eluate) can be added to the fractionation device 1000 or 1200, respectively, to separate the biomolecule 122 of interest from the contaminant(s) 1023 at a high-throughput production scale.

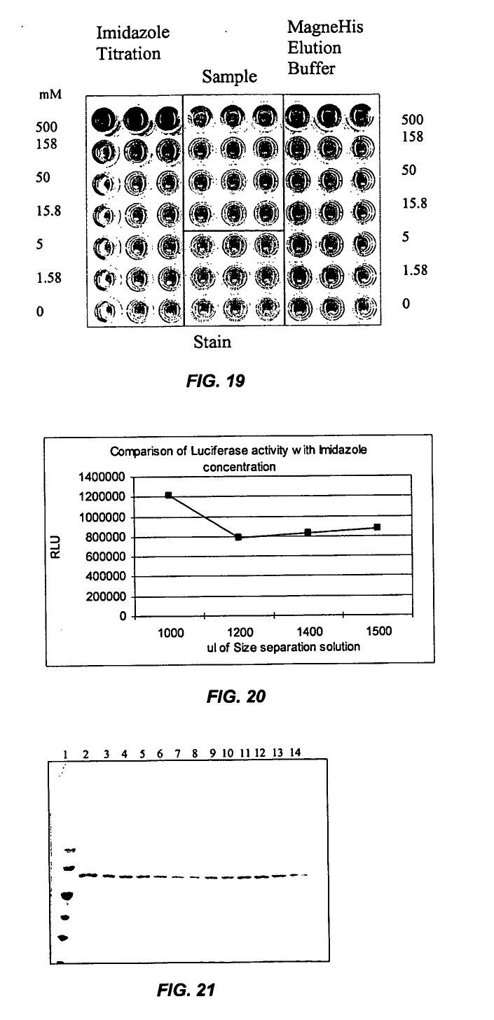

Working Example 8 and Prophetic Examples 9-15 relate generally to fractionation devices (alone or in combination biomolecule isolation apparatuses), systems and methods, and FIGS. 19-21 correspond to Example 8.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

The following working and prophetic examples are intended to be illustrative and not limiting.

### EXAMPLE 1

Isolation of His Tagged Proteins

Materials:

96-well plate, each well in the plate fitted with a 90 μm wire mesh as a filter that is sealed by an O-ring. Each well was predispensed with 4 mg of nickel-charged silica particles having a diameter of approximately 150 μm to approximately 200 μm. The silica particles used have an average pore size of 1000 Å, and a loading capacity of nickel of approximately 0.15 nmol/g of silica particle.
Wash buffer (100 mM HEPES, 10 mM imidazole)
Elution Buffer (100 mM HEPES, 500 mM imidazole)
10× Cell Lysis Buffer (0.5 M HEPES, 10% Triton X-100, 0.1 M imidazole, 6% octyl beta-D-thioglucopyranoside, 3% Tomah)
deionized water (dH20)
   Preparation of Cells:
JM109 cells containing the his tagged fusion protein, luciferase, (E. coli obtained from Promega Corporation, Madison, Wis., catalog no. L2001) were grown in a 96-well plate using 1 mL of LB media plus ampicillin (10 μg/mL of ampicillin). The 96-well plate was covered and shaken overnight at 37° C. The cultures were grown to an optical density (OD) at 600 nm of between 0.4 and 0.6 and then induced for protein expression.
IPTG induction: IPTG was added to obtain a final concentration of 1 mM and incubated at 37° C. for three hours, or for 25° C. overnight. Cell cultures had a final OD of less than or equal to 6. Generally, growing the cells overnight at 25° C. achieves an OD of less than or equal to 6. As a result, measuring the OD is optional.
5 mL of the induced cultures are pelleted by centrifugation using a 15 mL screw-cap centrifuge tube. When 1 mL of culture was used, the cells were directly lysed and no centrifugation was used.
The media was carefully decanted and the cells were resuspended by vigorously vortexing in 0.9 mL of dH₂O.
0.1 mL of Cell Lysis Buffer was added to the resuspended cells and mixed by gently swirling the mixture.
The resuspended, lysed, and buffered cells were incubated at room temperature (i.e., approximately 25° C.) for approximately 20 min. and mixed every 5 min. Care was taken to prevent excess frothing of the cell mixture.
   Isolation of His Tagged Proteins:
The 96-well plate was tapped on the benchtop to settle any silica particles that had been displaced during transport.
The cover on the 96-well plate was carefully removed.
The 96-well plate was placed in a vacuum manifold.
The 96-well plate was rehydrated by adding 1 mL of dH₂O per well, as needed.
Empty wells in the 96-well plate were covered tape to ensure effective vacuuming in later steps.
dH₂O was allowed to drain through an aperture in the bottom of each reservoir.
1 mL of the lysed cells were added while avoiding the generation of bubbles during transfer.
The cell lysate was allowed to slowly flow past the silica particles over a period of 5 min. (e.g., at a flow rate of approximately 0.5 mL/min.), ensuring effective binding between the His tagged protein and the nickel-charged silica solid phase.
A vacuum of approximately 10 in Hg was applied for 1 min. to dry the reservoirs.
1 mL of wash buffer was added to each reservoir. The 96-well plate was vacuumed for 1 min. using the vacuum manifold.
The wash sequence was repeated three times.
A vacuum was held for a total of 3 min. after the last wash to thoroughly dry the silica particles.
The 96-well plate was transferred to the elution manifold fitted with a fresh 96-well microtiter plate.
100 μL of elution buffer was added to each well and allowed to drain by gravity into a microtiter plate.
A vacuum of approximately 10 in Hg was applied for 2 min.
Eluted proteins were stored at −20° C.

### EXAMPLE 2

Isolation of Genomic DNA from Blood

Materials:

KFE8 Lysis Buffer
5.3M GTC (Guanidine Thiocyanate)
1% Triton® D X-100
1% CHAPS (3-[3-(Cholamidopropyl)dimethylammonio]-1-propanesulfonate)
0.1M EDTA ((Ethylenedinitrilo)tetraacetic acid), pH 8.0
1% Anti-Foam A

4/40 Wash
40% Isopropanol
4.2M Guanidine Hydrochloride

Alcohol Wash, Blood
25% Isopropanol
25% Ethanol
0.1M NaCl (Sodium Chloride)

Elution Buffer, Blood
10 mM Tris (Tris(hydroxymethyl)aminomethane), pH 8
0.1 M EDTA ((Ethylenedinitrilo)tetraacetic acid), pH 8
Vacuum, 96-Wells; Wizard® SV96 DNA Binding Plate retrofitted with 90 μm wire mesh as a filter that is sealed by an o-ring.
KFE8 Lysis Buffer (all samples)+100 mg silica particles per 800 μL; the silica particles used have a diameter of approximately 150 μm to approximately 200 μm and an average pore size of 1000 Å.
Isopropanol (IPA)
Vac-man® 96 (˜15 in. Hg)
   Isolation of Genomic DNA:
800 μL Lysis Buffer/Silica (100 mg) was added to 200 μL whole blood.
The 96-well plate was incubated at room temperature (RT; approximately 25° C.) or 68° C. for 10 min.
   For the RT samples, each sample was vortexed for 1 min. with the silica particles suspended.
   For the 68° C. samples, each sample was vortexed briefly after incubation to resuspend the silica.
The lysate was applied to each well in the 96-well plate. Care was taken to ensure that the silica particles were transferred.
Each well was washed twice with 1 mL of 4/40 Wash Solution.
Each well was washed twice with 1 mL of Alcohol Wash.
The 96-well plate was vacuum dried for 3 min. in the Vac-man® 96.
200 mL of elution buffer was added to each well and the 96-well plate was incubated at RT for 10 min.
A vacuum was applied for approximately 1 min. using the Vac-man® 96 to elute the genomic DNA into a collection plate.
The Vac-man® 96 was disassembled, and the eluted genomic DNA was stored at −20° C.

### EXAMPLE 3

Isolation of 6× His-Tagged Firefly Lucliferase Proteins from BL21 Cells

Materials:

96 well (deep well; 2 mL) BIO BLOCK™ 96-well plate (available from ABgene, catalog no. 0923)
Vacuum, 96 wells; Wizard® SV96 DNA Binding Plate retrofitted with 90 μm wire mesh as a filter that is sealed by an o-ring (“filter plate”)
DNase solution, prepared by adding the equivalent of 4 mL H₂O per vial of lyophilized DNase (available from Promega Corporation, Madison, Wis., catalog no. Z358)
Nickel-charged silica particles having a diameter of approximately 150 μm to approximately 200 μm. The silica particles used have an average pore size of 1000 Å, and a loading capacity of nickel of approximately 0.15 nmol/g of silica particle.
Lysis solution: FASTBREAK™ Cell Lysis Solution (available from Promega Corporation, Madison, Wis., catalog no. V5873)
Wash/Bind Buffer (available from Promega Corporation, Madison, Wis., catalog no. V851)
MAGNEHIS™ Elution Buffer (available from Promega Corporation, Madison, Wis., catalog no. V852B)
   Preparation of Cells:
1 mL of TB broth was placed in each well of the BIO BLOCK™ 96-well plate.
Each well was inoculated with BL21 (DE3) Star (available from Invitrogen Corporation) containing plasmid pJLC10, which encodes a his luciferase protein upon IPTG expression.
Cells were grown overnight at 37° C. and then induced using standard techniques.
After induction, 100 μL of the lysis solution was added to each well.
20 μL of DNase solution was added per well to decrease the viscosity of the solution.
   Isolation of His Luc Proteins:
90 μL of settled nickel charged silica particles (in H₂O) were added per well.
The BIO BLOCK™ 96-well plate was incubated for 30 min. at RT. Mixing was accomplished by pipetting every 5 min. using wide bore tips.
After incubation, the lysate and particles were transferred to the filter plate in 200 μL at a time, making sure to mix the particles into the lysate solution before each transfer.
A vacuum of 10 in Hg was applied for 30 seconds.
Each well was washed with 5×200 μL of the Wash/Bind Buffer.
A vacuum of 10 in Hg was applied for 1 min.
The filter plate was transferred to an elution setup, similar to that illustrated in FIG. 5C.
200 μL of the MAGNEHIS™ Elution Buffer was added to each well. The filter plate was incubated for 3 min. at room temperature (i.e., approximately 25° C.).
A vacuum of 10 in Hg was applied for 1 min. to elute the isolated proteins into a collection plate.
The elution setup was disassembled, and the eluted proteins were stored at −20° C.

### EXAMPLE 4

Automated Purification of 6× His Tagged Proteins

Materials:

96 well plate (available from Orachem, Philadelphia, Pa.) fitted with a 25 μm frit (“filter plate”)
Wash buffer (100 mM HEPES, 400 mM NaCl, 10 mM imidazole-HCl; brought to a pH of 7.5)
MAGNEHIS™ Elution Buffer (available from Promega Corporation, Madison, Wis., catalog no. V852B)
   Cell Culture Preparation:
6× His Firefly Luciferase expressed in BL-21 (DE3).
Cell were grown in Terrific Broth (TB) for overnight cultures.
5 ml of the overnight cultures were inoculated into 500 mL of TB.
Cultures were grown to an O.D_{.600} of 1.0-2.0 and induced with IPTG (final concentration 1 mM).
Cultures were grown overnight at 25° C. and harvested with a final O.D.₆₀₀ of 12.0.
Cultures were aliquotted and stored at −20° C. and thawed at time of use.
Cultures were diluted to O.D.₆₀₀ of 6.0, 4.0, 2.0, and 1.0 with fresh TB.
1 mL of these dilutions were placed into a BIO BLOCK™ 96-well plate (available from ABgene, catalog no. 0923).
   DNase Preparation:
One vial of lyophilized DNase (available from Promega Corporation, Madison, Wis., catalog no. Z385B) was resuspended in 80 μL of Nuclease Free Water (available from Promega Corporation, Madison, Wis., catalog no. P119C) and transferred to 1.24 mL of Nuclease Free Water.
808 μL of this dilution was added to 12.2 mL of FASTBREAK™ Lysis Reagent (available from Promega Corporation, Madison, Wis., catalog no. V882).
   Isolation of Proteins:
25 μL of HISLINK™ protein purification resin (available from Promega Corporation, Madison, Wis., catalog no. V8821; average particle size of approximately 90 μm) was used in this protocol as the solid phase.
Purification of the protein was performed on a BioMek 2000 (available from Beckman Coulter):
   The lysate and particles were transferred to the filter plate.
   The plate was suctioned for 10 s to pull the lysate past the filter (mesh).
   Wash buffer was added in 200 μL increments for a total of 1 mL and suctioned after the 200 μL, 600 μL and 1 mL applications.
   b 200 μL of the MAGNEHIS™ Elution Buffer was applied to the particles and allowed to react for 3 min.
   The particles were suctioned for 1 min. to collect the elutions.

FIG. 11 shows the results of Example 4. Lane 1: Protein marker (available from Promega Corporation, Madison, Wis., catalog no. V849A). Lane 2: Elution from 1.0 O.D.₆₀₀ culture. Lane 3: Elution from 2.0 O.D.₆₀₀ culture. Lane 4: Elution from 4.0 O.D.₆₀₀ of culture. Lane 5: Elution from 6.0 O.D.₆₀₀ of culture.

### EXAMPLE 5

Automated Purification of 6× His Tagged Proteins

Materials:

96 well plate (available from Orachem, Philadelphia, Pa.) fitted with a 90 μm wire mesh (“filter plate”)
Wash buffer (100 mM HEPES, 400 mM NaCl, 10 mM imidazole-HCl; brought to a pH of 7.5)
MAGNEHIS™ Elution Buffer (available from Promega Corporation, Madison, Wis., catalog no. V852B)
   Cell Culture Preparation:
6 His tagged MAP-kinase (MAPK) expressed in BL-21 (DE3) E. Coli cells.
Cells were grown in LB media for overnight cultures.
5 ml of the overnight cultures were inoculated into 500 mL of LB.
Cultures were grown to an O.D.₆₀₀ of 0.3 and induced with 100 mM IPTG final concentration 1 mM IPTG.
Cultures were grown at 37° C. and harvested with a final O.D.₆₀₀ of 1.14.
Cultures were aliquotted and stored at −20° C. and thawed at time of use.
1 mL of this culture was placed into a BIO BLOCK™ 96-well plate (available from ABgene, catalog no. 0923).
   Cell Culture Preparation:
6×-His tagged Calmodulin expressed in BL-21 (DE3).
Cells were grown in LB for overnight cultures.
5 ml of the overnight cultures were then inoculated into a 500 ml volume of LB.
Cultures were grown to an O.D.₆₀₀ of 0.4-0.6 and induced with 100 mM IPTG final concentration 1 mM IPTG.
Cultures were grown overnight at 25° C. and harvested with a final O.D.₆₀₀ of 1.79.
Cultures were aliquotted and stored at −20° C. and thawed at time of use.
1 ml of these dilutions were placed into the wells of a BIO BLOCK™ 96-well plate (available from ABgene, catalog no. 0923).
   DNase Preparation:
One vial of lyophilized DNase (available from Promega Corporation, Madison, Wis., catalog no. Z385A) was resuspended in 275 μL of Nuclease Free Water (available from Promega Corporation, Madison, Wis., catalog no. P119C) and then the entire vial was transferred to 4.0 mL of Nuclease Free Water (available from Promega Corporation, Madison, Wis., catalog no. P119C).
20 μL of this dilution was added to each well prior to purification.
   Isolation of Proteins: * 90 μL of Spherical SiNiADA silica particles (available from Silicycle, Quebec, Canada, catalog no. S74050 T; particle size ranging from approximately 120 μm to approximately 200 μm) was used in this protocol as the solid phase.
Purification of the protein was performed on a BioMek 2000 (available from Beckman Coulter):
   The lysate and particles were transferred to the filter plate.
   The plate was suctioned for 10 s to pull the lysate past the filter.
   Wash buffer was added in 200 μL increments for a total of 1 mL and suctioned after the 200 μL, 600 μL and 1 mL applications.
   200 μL of the MAGNEHIS™ Elution Buffer was applied to the particles and allowed to react for 3 min.
   The particles were suctioned for 1 min. to collect the elutions.

FIG. 12 illustrates the results of the 6×-His tagged Calmodulin experiment in Example 5. Lane 1: Elution from mesh plate after 500 μL of wash. Lane 2: Elution from mesh inserted plate after 750 μL of wash. Lane 3: Elution from mesh inserted plate after 1 mL of wash. Lane 4: Elution from mesh inserted plate after 4 mL of wash. Lane 5: Protein marker (available from Promega Corporation, Madison, Wis., catalog no. V849A) in a 96 well plate fitted with a frit (available from Innovative Microplates, catalog no. F20000).

FIG. 13 illustrates the results of the 6×-His tagged MAP-K experiment in Example 5. Lane 1: Elution from mesh plate after 500 μL of wash. Lane 2: Elution from mesh inserted plate after 750 μL of wash. Lane 3: Elution from mesh inserted plate after 1 mL of wash. Lane 4: Elution from mesh inserted plate after 4 mL of wash. Lane 5: Elution from a frit as a filter after 500 μL of wash. Lane 6: Protein Marker (available from Promega Corporation, Madison, Wis., catalog no. V849A).

### EXAMPLE 6

Manual Purification of 6× His Tagged Proteins

Materials:

96 well plate (available from Orachem, Philadelphia, Pa.) fitted with a 90 μm wire mesh (“filter plate”)
MAGNEHIS™ Wash buffer (available from Promega Corporation, Madison, Wis., catalog no. V851B)
MAGNEHIS™ Elution Buffer (available from Promega Corporation, Madison, Wis., catalog no. V852B)
   Cell Culture Preparation:
6× His Tagged Firefly Luciferase expressed in BL-21 (DE3).
Cells were grown in Terrific Broth (TB) for overnight cultures.
5 mL of the overnight cultures were then inoculated into 500 mL of TB.
Cultures were grown to an O.D.₆₀₀ of 1.0-2.0 and induced with IPTG (final concentration 1 mM).
Cultures were grown overnight at 25° C. and harvested with a final O.D.₆₀₀ of 12.0.
Cultures were aliquotted and stored at −20° C. and thawed at time of use.
Cultures were diluted to O.D.₆₀₀ of 2.0 with fresh TB.
10 mL of these dilutions were placed into 15 mL centrifuge tubes.
   DNase Preparation:
One vial of lyophilized DNase (available from Promega Corporation, Madison, Wis., catalog no. Z385A) was resuspended in 275 μL of Nuclease Free Water (available from Promega Corporation, Madison, Wis., catalog no. P119C) and then the entire vial was transferred to 4.0 mL of Nuclease Free Water (available from Promega Corporation, Madison, Wis., catalog no. P119C).
63.0 μL of this dilution was added to each tube.
   Isolation of Proteins:
1 mL of FASTBREAK™ Lysis Reagent (available from Promega Corporation, Madison, Wis., catalog no. V882) was added to the each tube.
The tube was mixed for 15 min.
1.0 mL of the lysate was aliquotted into 1.5 mL tubes and 90 μL of Spherical SiNiADA silica particles (available from Silicycle, Quebec, Canada, catalog no.

S74050 T; particle size ranging from approximately 120 μm to approximately 200 μm) was added to the tubes.
The tubes were mixed for 30 min. on a rotary mixer.
The lysate and particles were transferred to the filter plate.
The plate was suctioned for 10 s to pull the lysate past the filter.
Wash buffer was added in 200 μL increments for a total of 1 mL and suctioned after the 200 μL, 600 μL and 1 mL applications.
200 μL of the MAGNEHIS™ Elution Buffer was applied to the particles and allowed to react for 3 min.
The particles were suctioned for 1 min. to collect the elutions.

FIG. 14 illustrates the results of Example 6. Lane 1: Protein Marker (available from Promega Corporation, Madison, Wis., catalog no. V849A). Lane 2: Elution from plate with mesh filter after 1 elution. Lane 3: Elution from plate with mesh as a filter after 2 elutions.

### EXAMPLE 7

Manual Purification of 6× His Tagged Proteins

Materials:

96 well plate (available from Orachem, Philadelphia, Pa.) fitted with a 25 μm frit (“filter plate”)
Wash buffer (100 mM HEPES, 400 mM NaCl, 10 mM imidazole-HCl; brought to a pH of 7.5)
MAGNEHIS™ Elution Buffer (available from Promega Corporation, Madison, Wis., catalog no. V852)
   Cell Culture Preparation:
6× His Tagged Firefly Luciferase expressed in BL-21 (DE3).
Cells were grown in Terrific Broth (TB) for overnight cultures.
5 mL of the overnight cultures were then inoculated into 500 mL volume of TB.
Cultures were grown to an O.D.₆₀₀ of 1.0-2.0 and induced with IPTG (final concentration 1 mM).
Cultures were grown overnight at 25° C. and harvested with a final O.D.₆₀₀ of 12.0.
Cultures were aliquotted and stored at −20° C. and thawed at time of use.
Cultures were diluted to O.D.₆₀₀ of 4.0 with fresh TB.
1 mL of diluted culture were placed into a BIO BLOCK™ 96-well plate (available from Abgene, catalog no. 0923).
   DNase Preparation:
One vial of lyophilized DNase (available from Promega Corporation, Madison, Wis., catalog no. Z385A) was resuspended in 275 μL of Nuclease Free Water (available from Promega Corporation, Madison, Wis., catalog no. P119C) and then the entire vial was transferred to 4.0 mL of Nuclease Free Water (available from Promega Corporation, Madison, Wis., catalog no. P119C).
900 μL of this dilution was added to 13.0 mL of FASTBREAK™ Lysis Reagent (available from Promega Corporation, Madison, Wis., catalog no. V882).
   Isolation of Proteins:
100 μl of FASTBREAK™ Lysis Reagent/DNase solution was also added to each well.
25 μL of HISLINK™ protein purification resin (available from Promega Corporation, Madison, Wis., catalog no. V8821; average particle size of approximately 90 μm) was added to each of the wells.
The solutions were then mixed for 30 min. manually.
The lysate and particles were transferred to the filter plate.
The plate was suctioned for 10 s to pull the lysate past the filter.
Wash buffer was added in 200 μL increments for a total of 1 mL and suctioned after the 400 μL, 800 μL and 1 mL applications.
200 μL of the MAGNEHIS™ Elution Buffer (available from Promega Corporation, Madison, Wis., catalog no. V852B) was applied to the particles and allowed to react for 3 min. after which the particles were suctioned for 1 min. to collect the elutions.

FIG. 15 illustrates the results of Example 7. Lane 1: Protein Marker (available from Promega Corporation, Madison, Wis., catalog no. V849A). Lane 2: Elution using MAGNEHIS™ Elution Buffer.

### EXAMPLE 8

Purification of 6× Histidine Tagged Proteins and Removal of Imidazole from Histidine Tagged Proteins

Materials:

96 well plate (available from Orachem, Philadelphia, Pa.) fitted with a 25 μm frit (“filter plate”)
Wash buffer (100 mM HEPES, 10 mM imidazole-HCl; pH 7.5)
MAGNEHIS™ Elution Buffer (available from Promega Corporation, Madison, Wis., catalog no. V852)
   Cell Culture Preparation:
Polyhistidine Firefly Luciferase was expressed in BL-21 (DE3).
Cells were grown in Terrific Broth (TB) for overnight cultures.
5 mL of the overnight cultures were then inoculated into 500 mL volume of TB.
Cultures were grown to an O.D.₆₀₀ of 1.0-2.0 and induced with IPTG (final concentration 1 mM).
Cultures were grown overnight at 25° C. and harvested with a final O.D.₆₀₀ of 12.0.
Cultures were aliquotted and stored at −20° C. and thawed at time of use.
Cultures were diluted to O.D.₆₀₀ of 6.0, 4.0, 2.0, and 1.0 with fresh TB.
1 mL of these dilutions were placed into a BIO BLOCK™ 96-well plate (available from Abgene, catalog no. 0923).
   DNase Preparation:
One vial of lyophilized DNase (available from Promega Corporation, Madison, Wis., catalog no. Z385B) was resuspended in 80 μL of Nuclease Free Water (available from Promega Corporation, Madison, Wis., catalog no. P119C) and then the entire vial was transferred to 1.24 mL of Nuclease Free Water (available from Promega Corporation, Madison, Wis., catalog no. P119C).
808 μL of this dilution was added to 12.2 mL of FASTBREAK™ Lysis Reagent (available from Promega Corporation, Madison, Wis., catalog no. V882).
   Isolation of Proteins:
100 μL of HISLINK™ protein purification resin (available from Promega Corporation, Madison, Wis., catalog no. V8821; average particle size of approximately 90 μm) was used in this protocol as the solid phase.
Purification of the protein was performed on a BioMek 2000 (available from Beckman Coulter):
   The lysate and particles were transferred to the filter plate.
   The plate was suctioned for 10 s to pull the lysate past the filter.
   Wash buffer was added in 200 μL increments for a total of 1 mL and suctioned after the 200 μL, 600 μL and 1 mL applications.
   200 μL of the MAGNEHIS™ Elution Buffer was applied to the particles and allowed to react for 3 min.
   The particles were suctioned for 1 min. to collect the elutions.
   The elutions were pooled and mixed.
      Preparation of the Fractionation Solid Phase (i.e. Separation Gel):
A 5% solution was made using SEPHADEX® G-25 separation particles (available from Pharmacia/LKB, code no. 17-0032-01, Lt. 202722).
2 g of SEPHADEX® G-25 separation particles were added to 40.0 mL of Nanopure Water. This solution was mixed to resuspend the particles and to keep them suspended during the process of aliquotting.
The 5% solution was aliquotted into a filter plate in triplicates in the following volumes: 1.0, 1.2, 1.4 and 1.5 mL. This titration was completed in two rows of wells, and allowed to settle for approximately 1 hour.
The wells of the filter plate that were not being utilized were covered with an adhesive seal during the experiment.
The liquid in the wells of the filter plate was then suctioned through the wells by vacuum pressure.
The wells were then washed two times with 1 mL of Nanopure Water. The final wash was suctioned through by vacuum pressure.
   Removal of the Imidazole from the Polyhistidine Tagged Protein:
200 μL of the isolated polyhistidine tagged Luciferase was added to each well in one row of the SEPHADEX® G-25 separation particles titration.
200 μL of the MAGNEHIS™ Elution Buffer was added to each well in the second row of the SEPHADEX® G-25 separation particles titration. The filter plate was then suctioned for less than 15 seconds and the flow-through collected.
   Imidazole Detection:
A standard titration of imidazole HCl (1 M solution, pH 7.5) (available from Sigma, catalog no. 3386) was completed with the final concentration of 500, 158, 50, 15.8, 5, 1.58 and 0 mM.
A second dilution standard of the MAGNEHIS™ Elution Buffer was performed for the same final concentration of imidazole HCl in them.
150 μl of each of these titrations were placed into a clear 96 well plate (available from Fisher Scientific, catalog no. 12 565 501), in triplicate.
150 μl of the flow-through samples from the MAGNEHIS™ Elution Buffer was placed into different wells on the same plate.
150 μl of COOMASSIE PLUS™ Protein Assay Reagent (available from Pierce, product no. 1856210) was then added to the dilutions and the samples.
The samples were then mixed for 10 s on a plate shaker, and then read on a SpectraMax spectrophotometer (available from Molecular Devices) at 595 nm.

FIG. 19 illustrates the analysis of the flow-throughs that was completed by placing 150 μl of the samples into the wells of a clear plate.

Luciferase Activity Assay:

100 μL of the flow-throughs were removed and placed into the wells of a white 96 well plate (available from Costar, catalog no. 3912).
100 μl of BRIGHT GLO™ (BRIGHT GLO™ Luciferase Assay System, Promega Corporation, Catalog #E261) was added to the wells, and the samples were mixed for 10 s. The luminescence was read on an ORION plate reader (available form Berthold DS).
The results of the BRIGHT GLO™ assay are illustrated in FIG. 20.
   Gel Analysis of the Flow-through Containing Polyhisitidine Tagged Luciferase:
50 μl of the flow-through which contained polyhisitidine tagged luciferase was placed into a 0.5-mL centrifugation tube and mixed with 20 μL of gel running dye.
This mixture was heated at 95° C. for 5 min.
12 μL of the mixture was loaded onto a 4-20% Tris-Glycine gel (available from Invitrogen, catalog no. EC60355).
The gel was stained using SIMPLYBLUE™ Safe Stain (available from Invitrogen, catalog no. LC6065).
The electrophoretic gel is shown in FIG. 21. Lane 1: Molecular weight marker; Lane 2: Initial source of purified polyhistidine tagged Luciferase; Lanes 3-5: flow-throughs over 1.0 mL of 5% solution; Lanes 6-8: flow-throughs over 1.2 mL of 5% solution; Lanes 9-11: flow-throughs over 1.4 mL of 5% solution; Lanes 12-14: flow-throughs over 1.5 mL of 5% solution.

### EXAMPLE 9

Separation of Elution Molecules and Recombinant Proteins

The fractionation devices of the present invention used in the removal of molecules used in the elution of recombinant proteins by affinity chromatography. A fractionation multi-well plate would include a size exclusion fractionation device, which would enable the separation of proteins from contaminant elution molecules.

Fusion tags used in the isolation of recombinant proteins would include, without limitation, at least one of polyhistidine tagged proteins, metal affinity tags, GST fusion tags, thioredoxin tags biotinylated tags, streptavidin tags, and combinations thereof.

The elution molecules would include, without limitation, at least one of imidazole (for polyhistidine, GST or thioredoxin tagged proteins), EDTA (for polyhistidine, GST or thioredoxin tagged proteins), low pH (for polyhistidine, GST or thioredoxin tagged proteins), glutathione (for GST tagged proteins), biotin (for streptavidin binding tags), streptavidin tags (for biotinylated binding tags) and combinations thereof.

The removal of elution molecules from the eluted recombinant proteins would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein would be substantially free of contaminating elution molecules and could be used in downstream applications, including, without limitation, at least one of functional assays, interaction analysis, structural analysis, and combinations thereof.

### EXAMPLE 10

Separation of Elution Molecules from Phosphorylated Proteins

The fractionation devices of the present invention used in the removal of molecules used in the elution of phosphorylated proteins by metal affinity chromatography. Metal affinity particles would be used for the isolation of phosphorylated proteins. For example, Fe+++ or Ga+++ attached particles could be used. A fractionation multi-well plate would include a size exclusion fractionation device, which would enable the separation of proteins from contaminant elution molecules.

The elution molecules for eluting phosphorylated proteins would include, without limitation, at least one of ammonium hydroxide, or sodium hydroxide, alone or in combination with acetonitrile or TFA.

The removal of elution molecules from the eluted phosphorylated proteins would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein would be substantially free of contaminating elution molecules and could be used in downstream applications, including, without limitation, at least one of functional assays, interaction analysis, quantitation, structural analysis, and combinations thereof.

### EXAMPLE 11

Separation of Salts and Recombinant Proteins

The fractionation devices of the present invention used in the removal of salts present in elutions of recombinant proteins (e.g., for mass spectrometry analysis). A fractionation multi-well plate would include a size exclusion fractionation device, which would enable the separation of proteins from salts.

Fusion tags used in the isolation of recombinant proteins would include, without limitation, at least one of polyhistidine tagged proteins, metal affinity tags, GST fusion tags, thioredoxin tags, biotinylated tags, streptavidin tags, and combinations thereof.

The elution molecules would include, without limitation, at least one of imidazole (for polyhistidine, GST or thioredoxin tagged proteins), EDTA (for polyhistidine, GST or thioredoxin tagged proteins), low pH (for polyhistidine, GST or thioredoxin tagged proteins), glutathione (for GST tagged proteins), biotin (for streptavidin binding tags), streptavidin tags (for biotinylated binding tags) and combinations thereof.

The removal of salts from the eluted recombinant proteins would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein would be substantially free of contaminating salts and could be used in mass spectrometry analysis.

### EXAMPLE 12

Separation of Dyes (or Labels) and Recombinant Proteins

The fractionation devices of the present invention used in the removal of molecules used in covalent or non-covalent labeling of recombinant proteins by affinity chromatography.

A fractionation multi-well plate would include a size exclusion fractionation device, which would enable the separation of proteins and dyes (or labels).

The molecules used for the covalent or non-covalent labeling of recombinant proteins would include, without limitation, at least one of protein dyes (e.g., COOMASSIE BLUE™ dye, available from Pierce), fluorescent dyes, other ligand molecules, and combinations thereof.

The removal of these molecules from the eluted recombinant proteins would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein would be substantially free of contaminating dyes or labels and could be used in downstream applications, including, without limitation, at least one of functional assays, quantitation, and combinations thereof.

### EXAMPLE 13

Separation of Elution Molecules and Recombinant Proteins in Between Steps of a Two Step or Tandem Affinity Purification

The fractionation devices of the present invention used in the removal of molecules used in removal of elution molecules from a first elution to allow binding of the isolated protein to a subsequent solid phase.

(1) A nickel-charged solid phase of a first specific chemistry (e.g., IDA-nickel) would be used to isolate a desired polyhistidine tagged protein, and a second nickel-charged solid phase of a second specific chemistry (e.g., NTA-nickel, available from Qiagen or Promega) would be used to further isolate the desired polyhistidine tagged protein to achieve a highly purified protein. The fractionation devices of the present invention are used to remove the imidazole from the first elution to allow binding of the isolated protein to the second nickel-charge solid phase.

(2) Two different tags would be used in a two step or tandem isolation procedure. A protein would be tagged with two different tags: GST and polyhistidine. The tandem affinity purification would be applied in at least one of the following ways:
(a) First, elute the protein with one elution molecule, such as imidazole for polyhistidine tag. Subject this elution to a fractionation multi-well plate, or similar structure, that includes fractionation devices of the present invention to remove the imidazole. The resulting protein would then be subjected to GST purification where glutathione is used for the elution. A fractionation multi-well plate that includes fractionation devices of the present invention could also be used to remove the glutathione.
(b) GST-tagged proteins would be purified using metal affinity purification, such as a nickel-charged solid phase. In this case, the protein is isolated using the metal-charged solid phase. The elution from that isolation is then subjected to a fractionation multi-well plate, or similar structure, that includes fractionation devices of the present invention. The elution from the fractionation multi-well plate would then be purified used a second metal-charged solid phase.

### EXAMPLE 14

Separation of Metal and Recombinant Proteins

The fractionation devices of the present invention used in the removal of metal present in elutions of recombinant proteins by affinity chromatography. A fractionation multi-well plate would include a size exclusion fractionation device, which would enable the separation of proteins from the contaminating metal in the elution.

The removal of metal from the eluted recombinant proteins would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein would be substantially free of contaminating metal and could be used in downstream applications, including, without limitation, at least one of functional assays, quantitation, and combinations thereof.

### EXAMPLE 15

Separation of Endotoxins and Purified Recombinant Proteins or Purified DNA/RNA

The fractionation devices of the present invention used in the removal of endotoxins present in elutions of recombinant proteins or in purified DNA/RNA. A fractionation multi-well plate would include a size exclusion fractionation device, which would enable the separation of proteins or DNA/RNA from the contaminating endotoxins.

Endotoxins would include, without limitation, lipopolysaccharides associated with the outer membrane of Gram-negative bacteria, such as E. coli, Salmonella, Shigella, Pseudomonas, Neisseria, Haemophilus, and other leading pathogens.

The removal of endotoxins from the eluted recombinant proteins, or the purified DNA/RNA, would be achieved by passing the elutions through a fractionation multi-well plate that includes fractionation devices of the present invention. This would be achieved using the contaminant removal system 900 illustrated in FIG. 16 and described above, or the contaminant removal system 1100 illustrated in FIG. 18 and described above, and either vacuum, pressure or centrifugal force.

The resulting isolated protein or DNA/RNA would be substantially free of contaminating endotoxins. The isolated protein could be used in downstream applications, including, without limitation, at least one of functional assays, interaction analysis, structural analysis, and combinations thereof.

### EXAMPLE 16

Studying Protein-Protein Interactions

The fractionation devices of the present invention may be used to study protein-protein interactions.

For example, a lysate containing a tagged fusion protein (e.g., 6× histidine or GST tagged) is prepared. The lysate may be combined with a test protein and incubated for a certain period of time. Isolation of interacting recombinant protein complexes is by passing the lysate through an affinity purification multiwell plate followed by fractionation on a multi-well plate fractionation devices according to the present invention (e.g., the the contaminant removal system 900 illustrated in FIG. 16 and described above, or the contaminant removal system 1100 illustrated in FIG. 18 and described above).

Alternatively, a lysate containing a tagged fusion protein is first passed through an affinity purification multiwell plate and the eluate is transferred to a fractionation device the present invention (e.g., the contaminant removal system 900 illustrated in FIG. 16 and described above, or the contaminant removal system 1100 illustrated in FIG. 18 and described above). The purified tagged fusion protein is then contacted with a test protein and incubated for a certain period of time. Further isolation of tagged protein complexed with the test protein may be achieved by passing this sample through a fractionation devices of the present invention

### EXAMPLE 17

Studying Biomolecular Interactions

The fractionation devices of the present invention may be used to study the interaction of proteins with other molecules including, for example, small molecules, small molecules, nucleic acids, lipids, or carbohyrdates.

A lysate containing a tagged fusion protein (e.g., 6× histidine or GST tagged) is mixed with the test molecule and incubated for a certain period of time. Complexes of the protein and test molecule are isolated by passing the lysate through an affinity purification multiwell plate. The eluate is fractionated using a fractionation device of the present invention (e.g., the contaminant removal system 900 illustrated in FIG. 16 and described above, or the contaminant removal system 1100 illustrated in FIG. 18 and described above).

Alternatively, the lysate containing a tagged fusion protein is first passed through the affinity purification multiwell plate and the eluate is using a fractionation device of the present invention (e.g., the contaminant removal system 900 illustrated in FIG. 16 and described above, or the contaminant removal system 1100 illustrated in FIG. 18 and described above). The purified protein is them contacted with a test molecule and incubated for a certain period of time. Optionally, any recombinant protein-small molecule complexes present may be isolated by passing the sample through a fractionation devices of the present invention.

Various aspects of the invention are set forth in the following claims.

1. A method for isolating a biomolecule from a sample, the sample comprising the biomolecule and a contaminant, the method comprising:
providing a unitary device including a plurality of fractionation devices, each fractionation device including a reservoir, the reservoir comprising a filter, the reservoir adapted to contain a solid phase, the solid phase adapted to separate the biomolecule from the contaminant, the filter adapted to inhibit passage of the solid phase therethrough while allowing passage of the sample therethrough; and
moving the sample past the solid phase in the reservoir to separate the biomolecule from the contaminant by fractionation to obtain an isolated biomolecule.

2. The method of claim 1, further comprising performing a downstream application with the isolated biomolecule.

3. The method of claim 2, wherein the downstream application includes at least one of a functional assay, an interaction analysis, a quantitation, a structural analysis, a mass spectrometry measurement, a NMR measurement, a crystallization trial, and a combination thereof.

4. The method of claim 1, wherein the contaminant includes at least one of an elution molecule, a salt, a dye, a label, a metal, an endotoxin, and combinations thereof.

5. The method of claim 4, wherein the elution molecule includes at least one of imidazole, EDTA, a low pH solution, glutathione, biotin, streptavidin, ammonium hydroxide, sodium hydroxide, and combinations thereof.

6. The method of claim 1, wherein the unitary device comprises at least one of a multi-well plate, a plurality of capillary columns, a plurality of pipette tips, a plurality of baskets, and combinations thereof.

7. The method of claim 1, wherein the solid phase comprises at least one of a gel filtration resin, an ion exchange resin, an affinity resin, and combinations thereof.

8. The method of claim 1, wherein the solid phase includes a gel filtration resin and fractionation includes size exclusion chromatography.

9. The method of claim 1, wherein the solid phase includes an ion exchange resin and fractionation includes exposing the second solid phase to a pH gradient.

10. The method of claim 1, wherein the solid phase includes an affinity ion exchange resin and fractionation includes exposing the second solid phase to a salt gradient.

11. The method of claim 1, wherein the sample comprises an eluate from an upstream isolation process.

12. The method of claim 11, wherein the upstream isolation process includes:
providing a complex biological material comprising the biomolecule and insoluble matter;
providing a plurality of first reservoirs, each first reservoir comprising a first filter, each first reservoir adapted to contain a first solid phase, the first solid phase adapted to capture the biomolecule;
adding the complex biological material to the first reservoir;
combining the complex biological material with the first solid phase;
removing the insoluble matter from the sample by passing the insoluble matter through the first filter, the first filter having an average pore size sufficiently small to substantially prevent the first solid phase from passing therethrough;
contacting the biomolecule and the first solid phase with an elution buffer to form an eluate comprising the biomolecule and a contaminant; and
passing the eluate through the first filter.

13. The method of claim 12, wherein passing the eluate through the first filter includes passing the eluate through the first filter directly into one of the plurality of fractionation devices.
